(19) United States
(12) Patent Application Publication
(10) Pub. No.: US 2010/0260766 A1
(43) Pub. Date: Oct. 14, 2010

(54) STABLE ANTIBODY FORMULATIONS

(76) Inventors: Arvind Srivastava, Stewartsville, NJ (US); Joel Goldstein, Flemington, NJ (US)

Correspondence Address: ELI LILLY & COMPANY, PATENT DIVISION, P.O. BOX 6288, INDIANAPOLIS, IN, 46206-6288, US

(21) Appl. No.: 12/528,882

(22) PCT Filed: Mar. 20, 2008
(86) PCT No.: PCT/US08/57718

§ 371 Date: Aug. 27, 2009

Related U.S. Application Data
(60) Provisional application No. 60/919,744, filed on Mar. 22, 2007.

(51) Int. Cl. A61K 39/395

(52) U.S. Cl. 424/142.1; 424/158.1

(57) ABSTRACT

The present invention provides formulations and methods for the stabilization of antibodies. In one embodiment, the invention provides the stable solution formulation of an IgG1 antibody that specifically binds to insulin-like growth factor-I receptor. In another embodiment, the invention provides methods of stabilization of IgG1 antibody that specifically binds to insulin-like growth factor-I receptor comprising lyophilizing an aqueous formulation of the antibody. The formulations can be lyophilized to stabilize the antibodies during processing and storage, and then the formulations can be reconstituted for pharmaceutical administration.

### CROSS-REFERENCE

This application claims the benefit of U.S. provisional patent application Ser. No. 60/919,744, filed Mar. 22, 2007, the contents of which are incorporated by reference herein in its entirety.

### FIELD OF THE INVENTION

The present invention is directed to methods and formulations for the stabilization of antibodies that bind insulin-like growth factor-I receptor.

### BACKGROUND OF THE INVENTION

Antibodies in liquid formulations are susceptible to a variety of chemical and physical processes including hydrolysis, aggregation, oxidation, deamidation, and fragmentation at the hinge region. These processes can alter or eliminate the clinical efficacy of therapeutic antibodies by decreasing the availability of functional antibodies, and by reducing or eliminating their antigen binding characteristics. The present invention addresses the need for stable formulations of monoclonal antibodies of the IgG1 subclass that are directed against insulin-like growth factor receptor (IGF-IR) and provides stable solution formulations and stable lyophilized formulations for these antibodies.

IGF-IR is a ubiquitous transmembrane tyrosine kinase receptor that is essential for normal fetal and post-natal growth and development. IGF-IR can stimulate cell proliferation, cell differentiation, changes in cell size, and protect cells from apoptosis. It has also been considered to be quasi-obligatory for cell transformation (reviewed in Adams et al., Cell. Mol. Life. Sci. 57:1050-93 (2000); Baserga, Oncogene 19:5574-81 (2000)). The IGF-IR is located on the cell surface of most cell types and serves as the signaling-molecule for growth factors IGF-I and IGF-II (collectively termed henceforth IGFs). IGF-IR also binds insulin, albeit at three orders of magnitude lower affinity than it binds to IGFs. IGF-IR is a pre-formed hetero-tetramer containing two alpha and two beta chains covalently linked by disulfide bonds. The receptor subunits are synthesized as part of a single polypeptide chain of 180 kd, which is then proteolytically processed into alpha (130 kd) and beta (95 kd) subunits. The entire alpha chain is extracellular and contains the site for ligand binding. The beta chain possesses the transmembrane domain, the tyrosine kinase domain, and a C-terminal extension that is necessary for cell differentiation and transformation, but is dispensable for mitogen signaling and protection from apoptosis.

IGF-IR is highly similar to the insulin receptor (IR), particularly within the beta chain sequence (70% homology). Because of this homology, recent studies have demonstrated that these receptors can form hybrids containing one IR dimer and one IGF-IR dimer (Pandini et al., Clin. Canc. Res. 5:1935-19 (1999)). The formation of hybrids occurs in both normal and transformed cells and the hybrid content is dependent upon the concentration of the two homodimer receptors (IR and IGF-IR) within the cell. In one study of 39 breast cancer specimens, although both IR and IGF-IR were over-expressed in all tumor samples; hybrid receptor content consistently exceeded the levels of both homo-receptors by approximately 3-fold (Pandini et al., Clin. Canc. Res. 5:1935-44 (1999)). Although hybrid receptors are composed of Ht and IGF-IR pairs, the hybrids bind selectively to IGFs, with affinity similar to that of IGF-IR, and only weakly bind insulin (Siddle and Soos, The IGF System. Humana Press. pp. 199-225. 1999). These hybrids therefore can bind IGFs and transduce signals in both normal and transformed cells.

A second IGF receptor, IGF-IIR, or mannose-6-phosphate (M6P) receptor, also binds IGF-II ligand with high affinity, but lacks tyrosine kinase activity (Oates et al., Breast Cancer Res. Treat. 47:269-81 (1998)). Because it results in the degradation of IGF-II, it is considered a sink for IGF-H, antagonizing the growth promoting effects of this ligand. Loss of the IGF-IIR in tumor cells can enhance growth potential through release of its antagonistic effect on the binding of IGF-II with the IGF-IR (Byrd et al., J. Biol. Chem. 274:24408-16 (1999)).

Endocrine expression of IGF-I is regulated primarily by growth hormone and produced in the liver, but recent evidence suggests that many other tissue types are also capable of expressing IGF-I. This ligand is therefore subjected to endocrine and paracrine regulation, as well as autocrine in the case of many types of tumor cells (Yu, H. and Rohan, J., J. Natl. Cancer Inst. 92:1472-89 (2000)).

Six IGF binding proteins (IGFBPs) with specific binding affinities for the IGFs have been identified in serum (Yu, H. and Rohan, J., J. Natl. Cancer Inst. 92:1472-89 (2000)). IGFBPs can either enhance or inhibit the action of IGFs, as determined by the molecular structures of the binding proteins as a result of post-translational modifications. Their primary roles are for transport of IGFs, protection of IGFs from proteolytic degradation, and regulation of the interaction of IGFs with IGF-IR. Only about 1% of serum IGF-I is present as free ligand, the remainder is associated with IGFBPs (Yu, H. and Rohan, J., J. Natl. Cancer Inst. 92:1472-89 (2000)).

Upon binding of ligand (IGFs), the IGF-IR undergoes autophosphorylation at conserved tyrosine residues within the catalytic domain of the beta chain. Subsequent phosphorylation of additional tyrosine residues within the beta chain provides docking sites for the recruitment of downstream molecules critical to the signaling cascade. The principle pathways for transduction of the IGF signal are mitogen activated protein kinase (MAPK) and phosphatidylinositol 3-kinase (PI3K) (reviewed in Blakesley et al., In: The IGF System. Humana Press. 143-163 (1999)). The MAPK pathway is primarily responsible for the mitogenic signal elicited following IGFs stimulation and PI3K is responsible for the IGF-dependent induction of anti-apoptotic or survival processes.

A key role of IGF-IR signaling is its anti-apoptotic or survival function. Activated IGF-IR signals PI3K and downstream phosphorylation of Akt, or protein kinase B. Akt can effectively block, through phosphorylation, molecules such as BAD, which are essential for the initiation of programmed cell death, and inhibit initiation of apoptosis (Datta et al., Cell 91:231-41 (1997)). Apoptosis is an important cellular mechanism that is critical to normal developmental processes (Oppenheim, Annu. Rev. Neurosci. 14:453-501 (1991)). It is a key mechanism for effecting the elimination of severely damaged cells and reducing the potential persistence of mutagenic lesions that may promote tumorigenesis. To this end, it has been demonstrated that activation of IGFs signaling can promote the formation of spontaneous tumors in a mouse transgenic model (DiGiovanni et al., Cancer Res. 60:1561-70 (2000)). Furthermore, IGF over-expression can rescue cells from chemotherapy induced cell death and may be an important factor in tumor cell drug resistance (Gooch et al., Breast Cancer Res. Treat. 56:1-10 (1999)). Consequently, modulation of the IGF signaling pathway has been shown to increase the sensitivity of tumor cells to chemotherapeutic agents (Benini et al., Clinical Cancer Res. 7:1790-97 (2001)).

A large number of research and clinical studies have implicated the IGF-IR and its ligands (IGFs) in the development, maintenance, and progression of cancer. In tumor cells, over-expression of the receptor, often in concert with over-expression of IGF ligands, leads to, potentiation of these signals and, as a result, enhanced cell proliferation and survival. IGF-I and IGF-II have been shown to be strong mitogens for a wide variety of cancer cell lines including prostate (Nickerson et al., Cancer Res. 61:6276-80 (2001); Hellawell et al., Cancer Res. 62:2942-50 (2002)) breast (Gooch et al., Breast Cancer Res. Treat. 56:1-10 (1999)), lung, colon (Hassan and Macaulay, Ann. Oncol. 13:349-56 (2002)), stomach, leukemia, pancreas, brain, myeloma (Ge and Rudikoff, Blood 96:2856-61 (2000), melanoma (All-Ericsson et al., Invest. Ophthalmol. Vis. Sci. 43:1-8 (2002)), and ovary (reviewed in: Macaulay, Br. J. Cancer 65:311-20 (1990)) and this effect is mediated through the IGF-IR. High circulating levels of IGF-I in serum have been associated with an increased risk of breast, prostate, and colon cancer (Pollak, Eur. J. Cancer 36:1224-28 (2000)). In a mouse model of colon cancer, increases in circulating IGF-I levels in viva led to a significant increase in the incidence of tumor growth and metastasis (Wu et al., Cancer Res. 62: 1030-35 (2002)). Constitutive expression of IGF-I in epidermal basal cells of transgenic mice has been shown to promote spontaneous tumor formation (DiGiovanni et al., Cancer Res. 60:1561-1570 (2000; Bol et al., Oncogene 14:1725-1734 (1997)). Over-expression of IGF-II in cell lines and tumors occurs with high frequency and may result from loss of genomic imprinting of the IGF-II gene (Yaginuma et al., Oncology 54:502-7 (1997)). Receptor over-expression has been demonstrated in many diverse human tumor types including lung (Quinn et al., J. Biol. Chem. 271:11477-83 (1996)), breast (Cullen et al., Cancer Res. 50: 48-53 (1990); Peyrat and Bonneterre, Cancer Res. 22:59-67 (1992); Lee and Yee, Biomed. Pharmacother. 49:415-21 (1995)), sarcoma (van Valen et al., J. Cancer Res. Clin. Oncol. 118:269-75 (1992); Scotlandi et al., Cancer Res. 56:4570-74 (1996)), prostate (Nickerson et al., Cancer Res. 61:6276-80 (2001)), and colon (Hassan and Macaulay, Ann. Oncol. 13:349-56 (2002)). In addition, highly metastatic cancer cells have been shown to possess higher expression of IGF-II and IGF-IR than tumor cells that are less prone to metastasize (Guerra et al., Int. J. Cancer 65:812-20 (1996)). A critical role of the IGF-IR in cell proliferation and transformation was demonstrated in experiments of IGF-IR knockout derived mouse embryo fibroblasts. These primary cells grow at significantly reduced rates in culture medium containing 10% serum and fail to transform by a variety of oncogenes including. SV40 Large T (Sell et al., Mol. Cell. Biol. 3604-12 (1994)). Recently it was demonstrated that resistance to the drug Herceptin in some forms of breast cancer may be due to activation of IGF-IR signaling in these cancers (Lu et al., J. Natl. Cancer Inst. 93:1852-57 (2001)). Over-expression or activation of IGF-IR may therefore not only be a major determinant in tumorigenicity, but also in tumor cell drug resistance.

Activation of the IGF system has also been implicated in several pathological conditions besides cancer, including acromegaly (Drange and Melmed. In: The IGF System. Humana Press. 699-720 (1999)), retinal neovascularization (Smith et al., Nature Med. 12:1390-95 (1999)), and psoriasis (Wraight et al., Nature Biotech. 18:521-26 (2000)). In the latter study, an antisense oligonucleotide preparation targeting the IGF-IR was effective in significantly inhibiting the hyperproliferation of epidermal cells in human psoriatic skin grafts in a mouse model, suggesting that anti-IGF-IR therapies may be an effective treatment for this chronic disorder.

A variety of strategies have been developed to inhibit the IGF-IR signaling pathway in cells; Antisense oligonucleotides have been effective in vitro and in experimental mouse models, as shown above for psoriasis. In addition, inhibitory peptides targeting the IGF-IR have been generated that possess anti-proliferative activity in vitro and in vivo (Pietrzkowski et al., Cancer Res. 52:6447-51 (1992); Haylor et al., J. Am. Soc. Nephrol. 11:2027-35 (2000)). A synthetic peptide sequence from the C-terminus of IGF-IR has been shown to induce apoptosis and significantly inhibit tumor growth (Reiss et al., J. Cell. Phys. 181:124-35 (1999)). Several dominant-negative mutants of the IGF-IR have also been generated which, upon over-expression in tumor cell lines, compete with wild-type IGF-IR for ligand and effectively inhibit tumor cell growth in vitro and in vivo (Scotlandi et al., Int. J. Cancer 101:11-6 (2002); Seely et al., BMC Cancer 2:15 (2002)). Additionally, a soluble form of the IGF-IR has also been demonstrated to inhibit tumor growth in vivo (D'Ambrosio et al., Cancer Res. 56:4013-20 (1996)). Antibodies directed against the human IGF-IR have also been shown to inhibit tumor cell proliferation in vitro and tumorigenesis in vivo including cell lines derived from breast cancer (Artega and Osborne; Cancer Res. 49:6237-41 (1989)), Ewing's osteosarcoma (Scotlandi et al., Cancer Res. 58:4127-31 (1998)), and melanoma (Furlanetto et al., Cancer Res. 53:2522-26 (1993)). Antibodies are attractive therapeutics chiefly because of they 1) can possess high selectivity for a particular protein antigen, 2) are capable of exhibiting high affinity binding, to the antigen, 3) possess long half-lives in vivo, and, since they are natural immune products, should 4) exhibit low in vivo toxicity (Park and Smolen. In: Advances in Protein Chemistry. Academic Press. pp:360-421 (2001)). Antibodies derived from non-human sources, e.g.: mouse, may, however, effect a directed immune response against the therapeutic antibody, following repeated application, thereby neutralizing the antibody's effectiveness. Fully human antibodies offer the greatest potential for success as human therapeutics since they would likely be less immunogenic than murine or chimeric antibodies in humans, similar to naturally occurring immuno-responsive antibodies. To this end, there is a need to develop stable formulations of high affinity human anti-IGF-IR monoclonal antibodies for therapeutic use.

### SUMMARY OF THE INVENTION

The present invention is directed to formulations and methods for the stabilization of antibody preparations. In one embodiment, the invention provides a stable solution (or liquid) formulation comprising an IgG1 antibody that specifically binds to insulin-like growth factor-I receptor and a buffer. In a further embodiment the antibody concentration in the liquid formulation ranges from about 5 mg/ml to about 30 mg/ml. Preferably, the antibody is IMC-A12 or IMC-2F8. More preferably, the antibody is IMC-A12.

In one embodiment, the stable antibody solution formulation contains a citrate buffer. In a further embodiment, the citrate buffer is at a concentration between about 5 and about 50 mM. In a further embodiment, the citrate buffer is at a concentration of about 10 mM.

In one embodiment, the stable antibody solution formulation contains glycine. In a further embodiment, the glycine concentration is about 75 mM to about 150 mM. In a further embodiment, the glycine concentration is about 100 mM.

In one embodiment, the stable antibody solution formulation contains NaCl. In a further embodiment, the NaCl is at a concentration of about 75 to about 150 mM. In a further embodiment, the NaCl is at a concentration of about 100 mM.

In one embodiment, the stable antibody solution formulation contains a surfactant. In a further embodiment, the surfactant is a polysorbate (TWEEN, a/k/a polyethylene-polypropylene glycol), such as polysorbate 20 or polysorbate 80. In a further embodiment, the surfactant is polysorbate 80 (TWEEN 80) at a concentration of about 0.001% to about 1.0% (weight per volume). In a further embodiment, the TWEEN 80 is at a concentration of about 0.01% (weight per volume).

In one embodiment, the stable antibody solution formulation has a pH of about 6.0 to about 7.0. In a further embodiment, the pH is about 6.0 to about 6.5. In a further embodiment, the pH is about 6.5.

In one embodiment, the stable antibody solution formulation comprises about 5 mg/ml IMC-A12; about 10 mM sodium citrate, about 100 mM glycine, about 100 mM NaCl, and about 0.01% TWEEN 80, wherein said formulation is at a pH of about 6.5.

In one embodiment, the invention provides a stable, lyophilized antibody formulation comprising an IgG1 antibody that specifically binds to insulin-like growth factor-I receptor, wherein the formulation is lyophilized. In one embodiment, the antibody is IMC-A12. In a further embodiment, the IMC-A12 concentration is 30 mg/ml prior to lyophilization.

In one embodiment, the stable, lyophilized antibody formulation contains a histidine buffer. In a further embodiment, the histidine concentration is about 10 mM to about 50 mM prior to lyophilization. In a further embodiment, the histidine concentration is about 10 mM prior to lyophilization. In a further embodiment, the buffer is about pH 6.5 prior to lyophilization.

In one embodiment, the stable, lyophilized antibody formulation contains a lyoprotectant. In a further embodiment, the lyoprotectant is a sugar. In a further embodiment, the lyoprotectant is trehalose. In a further embodiment, the trehalose concentration is about 4.6% prior to lyophilization. In one embodiment the ratio of the trehalose concentration to the antibody concentration is between about 200 and about 1000 prior to lyophilization. In a further embodiment the ratio of the trehalose concentration to the antibody concentration is about 600 prior to lyophilization.

In one embodiment, the stable, lyophilized antibody formulation contains a bulking agent. In a further embodiment, the bulking agent is mannitol or glycine.

In one embodiment, the stable, lyophilized antibody formulation comprises about 30 mg/ml IMC-A12, about 10 mM histidine, and about 4.6% trehalose (weight/volume), wherein said formulation is at about pH 6.5, and wherein concentrations and pH are prior to lyophilizing.

### BRIEF DESCRIPTION OF THE FIGURES

FIG. 1 shows the variation of melting temperature (Tm1) as a function of pH in solution formulations of IMC-A12. Thermal melting curves for IMC-A12 in experimental formulations (from Table 2) were assayed by Differential Scanning Calorimetry (DSC) in order to assess the transition temperature (Tm) for IMC-A 12 in the test conditions.

FIG. 2 shows the variation of percent loss due to the formation of insoluble aggregate as a function of pH in solution formulations of IMC-A12. Samples as described in Table 2 including 5 mL of IMC-A12 at 5 mg/mL in 27.5 mL glass vials were agitated at 300 RPM at room temperature for 72 hours. Percent loss was analyzed by SEC-HPLC.

FIG. 3 shows variation of percent monomer as a function of pH in solution formulations of IMC-A12. Samples as described in Table 2 including 5 mL of IMC-A12 at 5 mg/mL in 27.5 mL glass vials were agitated at 300 RPM at room temperature for 72 hours. Percent monomer was analyzed by SEC-HPLC.

FIG. 4 shows the variation of percent monomer as a function of pH at 40° C. in solution formulations of IMC-A12. IMC-A12 at 5 mg/mL in various pH buffers (listed in Table 2) was incubated at 40° C. for 3 weeks. The effect of pH on percent monomer was analyzed by SEC-HPLC.

FIG. 5 shows the variation of percent monomer as a function of pH at 50° C. in solution formulations of IMC-A12. IMC-A12 at 5 mg/mL in various pH buffers (Table 2) was incubated at 50° C. for 1 week. The effect of pH on percent monomer was analyzed by SEC-HPLC.

FIG. 6 shows the variation of percent monomer as a function of pH at −20° C. and −70° C. in solution formulations of IMC-A12. IMC-A12 at 5 mg/mL in various pH buffers (listed in Table 2) were incubated at −20° C. and −70° C. for three weeks. The effect of pH on percent monomer was analyzed by SEC-HPLC.

FIG. 7 shows a prediction profiler for DSC study of solution formulations of IMC-A12. The prediction profiler for the effect of buffer type, pH, TWEEN 80 concentration, NaCl concentration, and glycine concentration on transition temperature was studied. The protein concentration was 5 mg/mL and temperature ramping was from 5° C. to 95° C. at a scan rate of 1.5° C./min. The melting temperature corresponding to the main transition peak was fitted to a linear regression model to estimate the effect of tested variables.

FIG. 8 shows a prediction profiler for an agitation study of solution formulations of IMC-A12. The samples described in Table 3 with 5 mL of IMC-A12 at 5 mg/mL in 27.5 mL glass vial were agitated at 300 RPM on a platform shaker. The study was performed at room temperature for up to 72 hours. Solution turbidity and percent monomer were determined as a function of agitation time. The effects of tested variables on turbidity and percent monomer were estimated by fitting the response to a linear regression model using JMP software.

FIG. 9 shows the percent monomer remaining in solution formulations of IMC-A12 after 4 weeks of incubation at 40° C. IMC-A12 at 5 mg/mL in the Table 3 formulations was incubated at 40° C. for 4 weeks. Percent monomer for starting material and tested formulations after 4 weeks of incubation at 40° C. was analyzed by SEC-HPLC.

FIG. 10 shows percent monomer remaining in solution formulations of IMC-A12 after 2 weeks of incubation at 50° C. IMC-A12 at 5 mg/mL in the Table 3 formulations was incubated at 50° C. for 2 weeks. Percent monomer for starting material and tested formulations after 2 weeks of incubation at 50° C. was analyzed by SEC-HPLC.

FIG. 11 shows a prediction profiler for the real time accelerated temperature stability of solution formulations of IMC-A12. The prediction profiler for the effect of pH, NaCl concentration, glycine concentration, time, and temperature on percent monomer, percent aggregate, and percent degradent was studied.

FIG. 12 shows a comparison of solution turbidity of Citrate and PBS formulations of IMC-A12 as a function of agitation time. The samples containing IMC-A12 at 5 mg/mL in 27.5 mL glass vials were agitated at 300 RPM on a platform shaker. The study was performed at room temperature for up to 72 hours. Turbidity was assayed by absorbance at 350 nm using a Shimatzu 1601 biospec spectrophotometer.

FIG. 13 shows a comparison of percent loss IMC-A12 in Citrate and PBS formulations as a function of agitation time. The samples containing IMC-A12 at 5 mg/mL in 27.5 mL glass vials were agitated at 300 RPM on a platform shaker. The study was performed at room temperature for up to 72 hours. Percent material loss (due to the formation of insoluble aggregate) was measured by SEC-HPLC.

FIG. 14 shows a comparison of percent IMC-A12 monomer in Citrate and PBS formulations as a function of agitation time. The samples containing IMC-A12 at 5 mg/mL in 27.5 mL glass vials were agitated at 300 RPM on a platform shaker. The study was performed at room temperature for up to 72 hours. The percent monomer was analyzed by SEC-HPLC.

FIG. 15 shows a comparison of percent monomer for IMC-A12 in PBS and Citrate formulations as a function of incubation time at 40° C. The percent monomer was analyzed by SEC-HPLC.

FIG. 16 shows a comparison of percent aggregate for IMC-A12 in PBS and Citrate solution formulations as a function of incubation time at 40° C. The percent aggregate was analyzed by SEC-HPLC.

FIG. 17 shows a comparison of percent degradent for IMC-A12 in PBS and Citrate solution formulations as a function of incubation time at 40° C. Percent degradent was measured by SEC-HPLC

FIG. 18 shows SDS-PAGE (reduced) for IMC-A12 in PBS and Citrate solution formulations, following 3 months of incubation at 40° C. Reducing SDS-PAGE was run on a 4-20% tris-glycine gradient gel. Ten μg of sample was loaded per lane in a volume of 10 μl. The gel was stained with Coomassie blue. “Citrate” of Lane 4 is: mg/mL IMC-A12, 10 mM Citrate, 100 mM Glycine, 100 mM NaCl, 0.01% TWEEN 80, pH 6.5]. “PBS” of Lanes 2 and 3 is phosphate buffered saline [see Tables 3 and 4, below].

FIG. 19 shows SDS-PAGE (non-reduced) for IMC-A12 in PBS and Citrate solution formulations, following 3 months of incubation at 40° C. Non-reducing SDS-PAGE was run using a 4-20% tris-glycine gradient gel. Ten μg of sample was loaded in a volume of 10 μl. The gel was stained with Coomassie blue. “Citrate” of Lane 4 is: 5 mg/mL IMC-A12, 10 mM Citrate, 100 mM Glycine, 100 mM NaCl, 0.01% TWEEN 80, pH 6.5]. “PBS” of Lanes 2 and 3 is phosphate buffered saline [see Tables 3 and 4, below].

FIG. 20 shows an Isoelectric focusing (IEF) gel for IMC-A12 in PBS and Citrate solution formulations following 3 months of incubation at 40° C. IEF was performed using IsoGel® Agarose IEF plates with a pH range from 6.0 to 10.5. Test samples were buffer exchanged into milliQ water containing 0.5% TWEEN 80. The 10 μg sample was loaded in a volume of 10 μl. The gel was stained with Coomassie blue. “Citrate” of Lane 4 is: 5 mg/mL IMC-A12, 10 mM Citrate, 100 mM Glycine, 100 mM NaCl, 0.01% TWEEN 80, pH 6.5]. “PBS” of Lanes 2 and 3 is phosphate buffered saline [see Tables 3 and 4, below].

FIG. 21 shows the variation of percent monomer as a function of time at −20° C. IMC-A12 at 5 mg/mL in PBS or Citrate solution formulations were incubated at −20° C. for up to 3 months. Following the incubation percent monomer was analyzed by SEC-HPLC.

FIG. 22 shows variation of percent monomer as a function of incubation time at −70° C. IMC-A12 at 5 mg/mL in PBS or Citrate solution formulations were incubated at −70° C. for up to 3 months. Following the incubation, percent monomer was analyzed by SEC-HPLC.

FIG. 23 shows variation of percent monomer as a function of number of freeze-thaw cycles at −20° C. Freeze-thaw stability of IMC-A12 was evaluated by freezing the test sample to −20° C. in a freeze-dryer with a ramp rate of 1° C./min. The sample was allowed to incubate for 1 hour and thawed at 4° C. with a ramp rate of 1° C./min. The freeze-thaw process was repeated up to 15 times and the percent monomer was analyzed by SEC-HPLC.

FIG. 24 shows variation of percent monomer as a function of number of freeze-thaw cycles at −70° C. Freeze-thaw stability of IMC-A12 was evaluated by freezing the test sample to −70° C. in a freeze-dryer with a ramp rate of 1° C./min. The sample was allowed to incubate for 1 hour and thawed at 4° C. with a ramp rate of 1° C./min. The freeze-thaw process was repeated up to 15 times and the percent monomer was analyzed by SEC-HPLC.

FIG. 25 shows the variation of percent monomer as a function of buffer type, cryo- and lyo protectants, bulking agents, time, and temperature.

FIG. 26 shows the variation of percent aggregate as a function of buffer type, cryo- and lyo protectants, bulking agents, time, and temperature.

FIG. 27 shows the variation of percent degradent as a function of buffer type, cryo- and lyo protectants, bulking agents, time, and temperature.

FIG. 28 shows the variation of solution turbidity as a function of buffer type, cryo- and lye protectants, bulking agents, time, and temperature.

FIG. 29 shows the variation of percent monomer for lyophilized formulations of IMC-A12. The effect of incubation at 40° C. and 50° C. for 3 months on the percent monomer in lyophilized formulations 5, 6, 9 and 10 in Table 6 was examined. Percent monomer was analyzed by SEC-HPLC.

FIG. 30 shows the variation of percent aggregate for lyophilized formulations of IMC-A12. The effect of incubation at 40° C. and 50° C. for 3 months on the percent aggregate in lyophilized formulations 5, 6, 9 and 10 in Table 6 was analyzed. Percent aggregate was analyzed by SEC-HPLC.

FIG. 31 shows the variation of percent degradent for lyophilized formulations of IMC-A12. The effect of incubation at 40° C. and 50° C. for 3 months on the percent degradent in lyophilized formulations 5, 6, 9 and 10 in Table 6 was analyzed. Percent degradent was analyzed by SEC-HPLC.

FIG. 32 shows the variation of solution turbidity for lyophilized formulations of IMC-A12. The effect of incubation at 40° C. and 50° C. for 3 months on the turbidity of lyophilized formulations 5, 6, 9 and 10 in Table 6 was analyzed after reconstitution to 5 mg/mL with Mili-Q water. Turbidity was assayed by absorbance at 350 nm using a Shimatzu 1601 biospec spectrophotometer.

FIG. 33 shows the variation of percent monomer remaining after 4 months of incubation. Lyophilized IMC-A12 formulations from Table 7 were incubated at 4° C., 40° C. and 50° C. for up to 4 months. The lyophilized samples were reconstituted with Milli-Q water to 5 mg/mL and analyzed by SEC-HPLC to determine the remaining monomer percent.

FIG. 34 shows circular dichorism spectra of IMC-A12, before (dotted line) and after lyophilization (solid line). To ensure that the lyophilization process has not altered the secondary structure of A12, secondary structure of IMC-A12 before and after lyophilizing was examined by circular dichorism. IMC-A12 was diluted or reconstituted into miliQ water to 0.1 mg/mL, and the circular dichorism spectrums were collected using a Jasco 810 circular dichorism spectrophotometer.

FIG. 35 shows the variation of percent monomer as a function of time at 40° C. IMC-A12 solution formulations in PBS and citrate buffer, and IMC-A12 in the preferred lyophilized formulation were incubated at 40° C. for 4 months. The lyophilized samples were reconstituted in Milli-Q water and percent monomer was analyzed by SEC-HPLC.

FIG. 36 shows the variation of percent monomer as a function of time at 50° C. IMC-A12 solution formulations in PBS and citrate buffer, and IMC-A12 in the preferred lyophilized formulation were incubated at 50° C. for 4 months. The lyophilized samples were, reconstituted in Milli-Q water and percent monomer was analyzed by SEC-HPLC.

FIG. 37 shows the variation of percent aggregate as a function of time at 40° C. IMC-A12 solution formulations in PBS and citrate buffer, and IMC-A12 in the preferred lyophilized formulation were incubated at 40° C. for 4 months. The lyophilized samples were reconstituted in Milli-Q water, and percent aggregate was analyzed by SEC-HPLC.

FIG. 38 shows the variation of percent aggregate as a function of time at 50° C. IMC-A12 solution formulations in PBS and citrate buffer, and IMC-A12 in the preferred lyophilized formulation were incubated at 50° C. for 4 months. The lyophilized samples were reconstituted in Milli-Q water and percent aggregate was analyzed by SEC-HPLC.

FIG. 39 shows the variation of percent degradent as a function of time at 40° C. IMC-A12 solution formulations in PBS and citrate buffer, and IMC-A12 in the preferred lyophilized formulation were incubated at 40° C. for 4 months. The lyophilized samples were reconstituted in Milli-Q water, and percent degradent was analyzed by SEC-HPLC.

FIG. 40 shows the variation of percent degradent as a function of time at 50° C. IMC-A12 solution formulations in PBS and citrate buffer, and IMC-A12 in the preferred lyophilized formulation were incubated at 40° C. and 50° C. for 4 months. The lyophilized samples were reconstituted in Milli-Q water and percent degradent was analyzed by SEC-HPLC.

FIG. 41 shows SDS-page (reduced) analysis of 4 months of incubated samples. IMC-A12 solution formulations in PBS and citrate buffer, and IMC-A12 in the preferred lyophilized formulation were incubated at 4° C., 40° C. and 50° C. for 4 months. The lyophilized samples were reconstituted in Milli-Q water and 10 μg were loaded into a 4-20% Tris-glycine gel. The gel was stained with Coomassie blue.

### DETAILED DESCRIPTION OF THE INVENTION

Antibodies in liquid formulations are susceptible to a variety of chemical and physical processes including hydrolysis, aggregation, oxidation, deamidation, and fragmentation at the hinge region. These processes can alter or eliminate the clinical efficacy of therapeutic antibodies by decreasing the availability of functional antibodies, and by reducing or eliminating their antigen binding characteristics. The present invention addresses the need for stable formulations of monoclonal antibodies and provides a method and formulation for lyophilizing these antibodies.

In one embodiment, the invention provides a stable solution formulation (also referred to herein as a “liquid formulation”) comprising an IgG1 antibody that specifically binds to insulin-like growth factor-I receptor and a buffer. In a further embodiment the antibody is IMC-A12. In another embodiment the antibody is IMC-2F8.

IMC-A12 is a fully human monoclonal antibody of the IgG1 subclass that is directed against insulin-like growth factor-1 receptor (IGF-1R). The IMC-A12 antibody is disclosed in PCT publication WO/2005/016970 incorporated by reference herein in its entirety. The nucleotide and amino acid sequence of the heavy chain for IMC-A12 are represented in SEQ ID NOS:1 and 2, respectively. The nucleotide and amino acid sequence of the light chain for EMC-A12 are represented in SEQ ID NOS:3 and 4, respectively. Methods of treating bone cancer using IMC-A12 are disclosed in PCT publication WO/2006/138729 incorporated by reference herein in its entirety.

IMC-2F8 is a fully human monoclonal antibody of the IgG1 subclass that is also directed against insulin-like growth factor-1 receptor (IGF-1R). The IMC-A12 antibody is disclosed in PCT publication WO/2005/016970 incorporated by reference herein in its entirety. The nucleotide and amino acid sequence of the heavy chain for IMC-2F8 are represented in SEQ ID NOS:1 and 2, respectively. The nucleotide and amino acid sequence of the light chain for IMC-2F8 are represented in SEQ ID NOS:5 and 6, respectively.

Formulation screening was performed in order to determine the robustness of the initial formulation, Phosphate Buffered Saline (PBS) at pH 7.2. It was determined from screening studies that IMC-A12 in PBS is sensitive to aggregation, precipitation, degradation, hydrolysis and light. In addition to that, it could not pass the test for particulate matter for small volume injectable. An improved solution formulation consisting of 5 mg/mL IMC-A12, 10 mM Sodium Citrate, 100 mM Glycine, 100 mM NaCl and 0.01% TWEEN 80 at a pH of 6.5 was developed. The citrate formulation, unlike PBS formulations was particulate free and has improved stability.

In a further improvement, that minimizes hydrolysis that occurs at the hinge region, a freeze-dried formulation that contains 30 mg/mL IMC-A12, 10 mM Histidine pH 6.5, and 4.6% Trehalose. Hydrolysis was stopped for IMC-A12 in freeze-dried formulation.

The present invention provides solution formulations that reduce or eliminate degradation of the antibody. The formulations may comprise one or more of the following: a buffer at a specific pH, salts, surfactants, stabilizing agents, preservatives, reducing agents, and chelating agents.

The present invention provides formulations for the freeze-drying of antibodies, including functional fragments thereof, that are prone to non-enzymatic cleavage. The formulations may comprise additional elements such as stabilizing agents, surfactants, reducing agents, carriers, preservatives, amino acids, and chelating agents.

The present invention also provides methods of stabilizing an antibody composition comprising lyophilizing an aqueous formulation of an antibody in the presence of a lyoprotectant. The formulations may be lyophilized to stabilize the antibodies during processing and storage, and then reconstituted prior to pharmaceutical administration. Preferably, the antibody substantially retains its physical and chemical stability and integrity from production to administration. Various formulation components may be suitable to enhance stability according to the present invention, including buffers, surfactants, sugars, sugar alcohols, sugar derivatives, and amino acids. Various formulation properties may be suitable to enhance stability according to the present invention, including pH and concentration of formulation components.

According to the present invention, a buffer may be used to maintain the pH of the formulation. The buffer minimizes fluctuations in pH due to external variations. The formulations of the present invention contain one or more buffers to provide the formulations at a suitable pH, preferably about 6.0 to about 7.0, more preferably about 6.0 to about 6.5, and most preferably about 6.5. Exemplary buffers include, but are not limited to organic buffers generally, such as histidine, citrate, malate, tartrate, succinate, and acetate. In one embodiment the buffer concentration is about 5 mM to about 50 mM. In a further embodiment the buffer concentration is about 10 mM.

The formulations of the present invention may contain one or more stabilizing agents, which may help prevent aggregation and degradation of the antibodies. Suitable stabilizing agents include, but are not limited to polyhydric sugars, sugar alcohols, sugar derivatives, and amino acids. Preferred stabilizing agents include, but are not limited to aspartic acid, lactobionic acid, glycine, trehalose, mannitol, and sucrose.

The formulations of the present invention may contain one or more surfactants. Antibody solutions have high surface tension at the air-water interface. In order to reduce this surface tension, antibodies tend to aggregate at the air-water interface. A surfactant minimizes antibody aggregation at the air-water interface, thereby helping to maintain the biological activity of the antibody in solution. For example, adding 0.01% TWEEN 80 can reduce antibody aggregation in solution. When the formulation is lyophilized, the surfactant may also reduce the formation of particulates in the reconstituted formulation. In the lyophilized formulations of the present invention, the surfactant can be added to one or more of the pre-lyophilized formulation, the lyophilized formulation, and the reconstituted formulation, but preferably the pre-lyophilized formulation. For example, 0.01% TWEEN 80 can be added to the antibody solution before lyophilization. Surfactants include, but are not limited to polysorbate 20 (TWEEN 20), polysorbate 80 (TWEEN 80), polyethylene-polypropylene glycol (PLURONIC F-68, CAS #9003-11-6), and bile salts. In one embodiment, the surfactant concentration is about 0.001% to about 1.0%.

The lypohilization process can generate a variety of stresses that may denature proteins or polypeptides. These stresses include temperature decrease, ice crystal formation, ionic strength increase, pH changes, phase separation, removal of hydration shell, and concentration changes. Antibodies that are sensitive to the stresses of the freezing and/or drying process can be stabilized by adding one or more lyoprotectants. A lyoprotectant is a compound that protects against the stresses associated with lyophilization. Therefore lyoprotectants as a class include cryoprotectants, which just protect from the freezing process. One or more lyoprotectants may be used to protect from the stresses associated with lyophilization and may be, for example, a sugar such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and manmitol; propylene glycol; polyethylene glycol; Pluronics; and combinations thereof. Examples of preferred lyoprotectants include, but are not limited to the stabilizing agents and surfactants as described above.

The present invention provides stabilized formulations, which may be prepared through the process of lyophilization. Lyophilization is a stabilizing process in which a substance is first frozen and then the quantity of the solvent is reduced, first by sublimation (the primary drying process) and then desorption (the secondary drying process) to values that will no longer support biological activity or chemical reactions. In a lyophilized formulation, the hydrolysis, deamidation, oxidation and fragmentation reactions associated with solutions can be avoided or slowed significantly. A lyophilized formulation may also avoid damage due to short-term temperature fluctuations during shipping and allow for room temperature storage. The formulations of the present invention may also be dried by other methods known in the art such as spray drying and bubble drying. Unless otherwise specified, the formulations of the present invention are described in terms of their component concentrations as measured in the formulation before lyophilization.

In one embodiment, the present invention provides for methods and formulations to stabilize antibodies that are prone to non-enzymatic degradation, which may occur at the hinge region. Factors that may predispose an antibody to non-enzymatic cleavage include amino acid sequence, conformation and post-translational processing.

Determination that an antibody undergoes hydrolysis, aggregation, oxidation, deamidation, precipitation, and/or fragmentation at the hinge region may be accomplished by incubation of the antibody in an aqueous solution. Typically, the incubation is performed at elevated temperatures to shorten the duration of the study. For example, incubation for 3 months at 40° C. or 50° C. Following the incubation, the degradation products may be analyzed using size exclusion chromatography-high performance liquid chromatography (SEC-HPLC).

In addition, antibody formulations may be agitated to examine protective effects of the formulation components on mechanical stress-induced degradation, aggregation, and precipitation of the antibody.

Various analytical techniques known in the art can measure the antibody stability of a solution formulations or of reconstituted lyophilized formulation. Such techniques include, for example, determining (i) thermal stability using differential scanning calorimetry (DSC) to determine the main melting, temperature (Tm); (ii) mechanical stability using controlled agitation at room temperature; (iii) real-time isothermal accelerated temperature stability at temperatures of about −20° C., about 4° C., room temperature (about 23° C.-27° C.), about 40° C., and about 50° C.; (iv) solution turbidities by monitoring absorbance at about 350 nm and (v) the amount of monomer, aggregates and degradants using SEC-HPLC. Stability can be measured at a selected temperature for a selected time period.

In one embodiment, the lyophilized formulation provides a high concentration of the antibody upon reconstitution. In a further embodiment, the stable lyophilized formulation is reconstitutable with a liquid to form a solution with an antibody concentration about 1-10 times higher than the antibody concentration of the formulation before lyophilization. For instance, in one embodiment, the lyophilized formulation is reconstituted with 1 mL of water or less to obtain a particle-free reconstituted formulation with an antibody concentration of about 50 mg/mL to about 200 mg/mL.

Naturally occurring antibodies typically have two identical heavy chains and two identical light chains, with each light chain covalently linked to a heavy chain by an interchain disulfide bond. Multiple disulfide bonds further link the two heavy chains to one another. Individual chains can fold into domains having similar sizes (110-125 amino acids) and structures, but different functions. The light chain can comprise one variable domain (V_{L}) and/or one constant domain (C_{L}). The heavy chain can also comprise one variable domain (V_{H}) and/or, depending on the class or isotype of antibody, three or four constant domains (C_{H}1, C_{H}2, C_{H}3 and C_{H}4). In humans, the isotypes are IgA, IgD, IgE, IgG, and IgM, with IgA and IgG further subdivided into subclasses or subtypes and (IgA₁₋₂ and IgG₁₋₄).

Generally, the variable domains show considerable amino acid sequence variability from one antibody to the next, particularly at the location of the antigen-binding site. Three regions, called hypervariable or complementarity-determining regions (CDRs), are found in each of V_{L} and V_{H}, which are supported by less variable regions called framework variable regions.

The portion of an antibody consisting of V_{L} and V_{H} domains is designated Fv (fragment variable) and constitutes the antigen-binding site. Single chain Fv (scFv) is an antibody fragment containing a V_{L} domain and a V_{H} domain on one polypeptide chain, wherein the N terminus of one domain and the C terminus of the other domain are joined by a flexible linker (see, e.g., U.S. Pat. No. 4,946,778 (Ladner et al.); WO 88/09344, (Huston et al.). WO 92/01047 (McCafferty et al.) describes the display of scFv fragments on the surface of soluble recombinant genetic display packages, such as bacteriophage.

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one V_{H} and one V_{L} domain covalently linked by a first peptide linker, can be covalently linked by at least one or more peptide linker to form a multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of amino acid residues is about one hundred.

Two single chain antibodies can be combined to form a diabody, also known as a bivalent dimer. Diabodies have two chains and two binding sites, and can be monospecific or bispecific. Each chain of the diabody includes a V_{H} domain connected to a V_{L} domain. The domains are connected with linkers that are short enough to prevent pairing between domains on the same chain, thus driving the pairing between complementary domains on different chains to recreate the two antigen-binding sites.

Three single chain antibodies can be combined to form triabodies, also known as trivalent trimers. Triabodies are constructed with the amino acid terminus of a V_{L} or V_{H} domain directly fused to the carboxyl terminus of a V_{L} or V_{H} domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

Fab (Fragment, antigen binding) refers to the fragments of the antibody consisting of V_{L} C_{L} V_{H} and C_{H}1 domains. Those generated following papain digestion simply are referred to as Fab and do not retain the heavy chain hinge region. Following pepsin digestion, various Fabs retaining the heavy chain hinge are generated. Those divalent fragments with the interchain disulfide bonds intact are referred to as F(ab′)₂, while a monovalent Fab′ results when the disulfide bonds are not retained. F(ab′)₂ fragments have higher avidity for antigen that the monovalent Fab fragments.

Fc (Fragment crystallization) is the designation for the portion or fragment of an antibody that comprises paired heavy chain constant domains. In an IgG antibody, for example, the Fc comprises C_{H}2 and C_{H}3 domains. The Fc of an IgA or an IgM antibody further comprises a C_{H4} domain. The Fc is associated with Fc receptor binding, activation of complement-mediated cytotoxicity, and antibody-dependent cellular-cytoxicity (ADCC). For antibodies such as IgA and IgM, which are complexes of multiple IgG like proteins, complex formation requires Fc constant domains.

Finally, the hinge region separates the Fab and Fc portions of the antibody, providing for mobility of Fabs relative to each other and relative to Fc, as well as including multiple disulfide bonds for covalent linkage of the two heavy chains.

Thus, antibodies of the invention include, but are not limited to, naturally occurring antibodies, bivalent fragments such as (Fab′)₂, monovalent fragments such as Fab, single chain antibodies, single chain Fv (scFv), single domain antibodies, multivalent single chain antibodies, diabothes, triabodies, and the like that bind specifically with antigens.

Antibodies, or fragments thereof, of the present invention, for example, can be monospecific or bispecific. Bispecific antibodies (BsAbs) are antibodies that have two different antigen-binding specificities or sites. Where an antibody has more than one specificity, the recognized epitopes can be associated with a single antigen or with more than one antigen. Thus, the present invention provides bispecific antibodies, or fragments thereof, that bind to two different antigens

Specificity of antibodies, or fragments thereof, can be determined based on affinity and/or avidity. Affinity, represented by the equilibrium constant for the dissociation of an antigen with an antibody (K_{d}), measures the binding strength between an antigenic determinant and an antibody-binding site. Avidity is the measure of the strength of binding between an antibody with its antigen. Avidity is related to both the affinity between an epitope with its antigen binding site on the antibody, and the valence of the antibody, which refers to the number of antigen binding sites of a particular epitope. Antibodies typically bind with a dissociation constant (K_{d}) of 10⁻⁵ to 10⁻¹¹ liters/mol. Any K_{d} less than 10⁻⁴ liters/mol is generally considered to indicate nonspecific binding. The lesser the value of the K_{d}, the stronger the binding strength between an antigenic determinant and the antibody binding site.

As used herein, “antibodies” and “antibody fragments” includes modifications that retain specificity for a specific antigen. Such modifications include, but are not limited to, conjugation to an effector molecule such as a chemotherapeutic agent (e.g., cisplatin, taxol, doxorubicin) or cytotoxin (e.g., a protein, or a non-protein organic chemotherapeutic agent). The antibodies can be modified by conjugation to detectable reporter moieties. Also included are antibodies with alterations that affect non-binding characteristics such as half-life (e.g., pegylation).

Proteins and non-protein agents may be conjugated to the antibodies by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990) for the conjugation of doxorubicin and those described by Arnon et al., Adv. Exp. Med. Biol. 303, 79-90 (1991) and by Kiseleva et al., Mol. Biol. (USSR)25, 508-514 (1991) for the conjugation of platinum compounds.

Antibodies of the present invention further include those for which binding characteristics have been improved by direct mutation, methods of affinity maturation, phage display, or chain shuffling. Affinity and specificity can be modified or improved by mutating CDRs and screening for antigen binding sites having the desired characteristics (see, e.g., Yang et al., J. Mol. Biol., 254: 392-403 (1995)). CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (see, e.g., Hawkins et al., J. Mol. Biol., 226: 889-896 (1992)). For example, phage display vectors containing heavy and light chain variable region genes can be propagated in mutator strains of E. coli (see, e.g., Low et al., J. Mol. Biol., 250: 359-368 (1996)). These methods of mutagenesis are illustrative of the many methods known to one of skill in the art.

Antibodies may also be modified to contain one or more amino acid substitutions in the Fc region that alter binding to Fc receptors thus increasing or decreasing effector functions such as antibody-dependant cell-mediated cytotoxicity and complement-dependant cytotoxicity.

Each domain of the antibodies of this invention can be a complete immunoglobulin domain (e.g., a heavy or light chain variable or constant domain), or it can be a functional equivalent or a mutant or derivative of a naturally-occurring domain, or a synthetic domain constructed, for example, in vitro using a technique such as one described in WO 93/11236 (Griffiths et al.). For instance, it is possible to join together domains corresponding to antibody variable domains, which are missing at least one amino acid. The important characterizing feature of the antibodies is the presence of an antigen binding site. The terms variable heavy and light chain fragment should not be construed to exclude variants that do not have a material effect on specificity.

Antibodies and antibody fragments of the present invention can be obtained, for example, from naturally occurring antibodies, or Fab or scFv phage display libraries. It is understood that, to make a single domain antibody from an antibody comprising a V_{H} and a V_{L} domain, certain amino acid substitutions outside the CDRs can be desired to enhance binding, expression or solubility. For example, it can be desirable to modify amino acid residues that would otherwise be buried in the V_{H}-V_{L} interface.

Further, antibodies and antibody fragments of the invention can be obtained by standard hybridoma technology (Harlow & Lane, ed., Antibodies: A Laboratory Manual, Cold Spring Harbor, 211-213 (1998), which is incorporated by reference herein) using transgenic mice (e.g., KM mice from Medarex, San. Jose, Calif.) that produce human immunoglobulin gamma heavy and kappa light chains. In a preferred embodiment, a substantial portion of the human antibody producing genome is inserted into the genome of the mouse, and is rendered deficient in the production of endogenous murine antibodies. Such mice may be immunized subcutaneously (s.c.) with part or all of target molecule in complete Freund's adjuvant.

The present invention also provides a method of treatment comprising administering a reconstituted formulation. The reconstituted formulations are prepared by reconstituting the lyophilized formulations of the present invention, for example with 1 mL water. The reconstitution time is preferably less than 1 minute. The concentrated reconstituted formulation allows for flexibility in administration. For example, the reconstituted formulation can be administered in a dilute form intravenously, or it can be administered in a more concentrated form by injection. A concentrated reconstituted formulation of the present invention can be diluted to a concentration that is tailored to the particular subject and/or the particular route of administration. Accordingly, the present invention provides methods of treatment comprising administering a therapeutically effective amount of an antibody to a mammal, particularly a human, in need thereof. The term administering as used herein means delivering the antibody composition of the present invention to a mammal by any method that can achieve the result sought. The reconstituted formulation can be administered, for example, intravenously or intramuscularly. In one embodiment, a concentrated reconstituted formulation is administered by injection.

Antibodies in the formulations of the present invention are preferably human. In one embodiment the composition of the present invention may be used to treat neoplastic diseases, including solid and non-solid tumors, for treatment of hyperproliferative disorders, for the treatment of obesity.

Therapeutically effective amount means an amount of antibody of the present invention that, when administered to a mammal, is effective in producing the desired therapeutic effect, such as reducing or neutralizing IGF-IR activity, inhibition of tumor growth, treating a non-cancerous hyperproliferative disease, treating obesity. Administration of the antibodies as described above can be combined with administration of other antibodies or any conventional treatment agent, such as an anti-neoplastic agent.

In an embodiment of the invention, the composition can be administered in combination with one or more anti-neoplastic agents. Any suitable anti-neoplastic agent can be used, such as a chemotherapeutic agent, radiation or combinations thereof. The anti-neoplastic agent can be an alkylating agent or an anti-metabolite. Examples of alkylating agents include, but are not limited to, cisplatin, cyclophosphamide, melphalan, and dacarbazine. Examples of anti-metabolites include, but not limited to, doxorubicin, daunorubicin, paclitaxel, irinotecan (CPT-11), and topotecan. When the anti-neoplastic agent is radiation, the source of the radiation can be either external (external beam radiation therapy—EBRT) or internal (brachytherapy BT) to the patient being treated. The dose of anti-neoplastic agent administered depends on numerous factors, including, for example, the type of agent, the type and severity tumor being treated and the route of administration of the agent. It, should be emphasized, however, that the present invention is not limited to any particular dose.

Equivalents of the antibodies, or fragments thereof, of the present invention also include polypeptides with amino acid sequences substantially the same as the amino acid sequence of the variable or hypervariable regions of the full-length IMC-A12 antibody provided herein. Substantially the same amino acid sequence is defined herein as a sequence with at least about 70%, preferably at least about 80%, and more preferably at least about 90% homology, as determined by the FASTA search method in accordance with Pearson and Lipman (Proc. Natl. Acad. Sci. USA 85, 2444-8 (1988)).

### EXAMPLES

The following examples further illustrate the invention, but should not be construed to limit the scope of the invention in any way. Detailed descriptions of conventional methods, such as those employed in the analysis of proteins can be obtained from numerous publications such as Current Protocols in Immunology (published by published by John Wiley & Sons). All references mentioned herein are incorporated in their entirety.

For all liquid formulation screening studies, the protein concentration was fixed at 5 mg/mL. A multi-component buffer consisting of 10 mM Sodium Phosphate, 10 mM Sodium Citrate, 10 mM Sodium Acetate, 10 mM L-Histidine and 125 mM Sodium Chloride was used to screen for the optimal pH. Buffer type, requirement for TWEEN 80, glycine concentration, and NaCl concentration were examined using a design of experiment approach (DOE, IMP software). Linear regression analysis was performed to determine the significance of tested variables. The predicted formulation was confirmed using a traditional one-factor-at-a-time methodology. The effect of the tested variables on thermal stability was examined using differential scanning calorimetry (DSC) and real-time isothermal studies. Controlled agitation at 300 rpm at room temperature was used as a test for mechanical stability. Photo stability of the liquid formulations was examined per ICH guidelines. Freeze-thaw stability was determined by freezing test samples to −20° C. and −70° C. and thawing at 4° C.

For lyophilized IMC-A12 formulations, buffer type, stabilizers, and bulking agents were examined, using design of experiments fractional factorial model at A12 concentration of 20 mg/mL. The concentration of IMC-A12, ratio of trehalose concentration to IMC-A12 concentration, and the concentration of TWEEN 80 was optimized using mixture design model. The predicted optimal freeze-dried formulation was compared with PBS and Citrate solution formulations using one-factor-at-a-time methodology. The effect of variables on thermal stability was examined by real-time isothermal studies. Photo stability of the lyophilized formulation was examined per ICH guidelines.

TABLE 1
Materials, Grade and Vendors

| MATERIALS | GRADE | VENDOR |
|---|---|---|
| IMC-A12 | 1278-116, 1278-151 | N/A |
| Sodium Citrate Dihydrate | USP | J. T. Baker |
| Citric Acid Anhydrous | USP | J. T. Baker |
| Sodium Acetate | USP | J. T. Baker |
| L-Histidine | USP | J. T. Baker |
| Sodium dibasic phosphate | USP | J. T. Baker |
| Sodium monobasic | USP | J. T. Baker |
| phosphate | | |
| NaCl | USP | J. T. Baker |
| Tween 80 | Multi compendia | J. T. Baker |
| Glycine | USP | J. T. Baker |
| Sucrose | Multi compendia | Ferro Pfanstiehl |
| Trehalose | Multi compendia | Ferro Pfanstiehl |
| Manitol | Multi compendia | J. T. Baker |

IMC-A12 for use in screening studies was prepared by buffer exchange into experimental buffers using 50K cut-off (YM 50) centriprep centrifugal filtration devices and an Allegra X-12R centrifuge (Beckman). The protein concentration was determined by absorbance at 280 nm using an extinction coefficient of 1.50 and the concentration adjusted to 0.5 mg/mL with the appropriate buffer. TWEEN 80 was added from a 10% (w/v) stock solution following protein concentration adjustments. IMC-A12 at 5 mg/mL in PBS formulation was used as a control. All samples were 0.22 μm filtered through a syringe filter (Durapore PVDF membrane).

The freeze-drying process was performed using Lyostar II-freeze-dryer. The product was loaded in to lyophilizer at room temperature. The shelf temperature was cooled to −50° C. with a cooling rate of 0.5° C./min. Soaking time at −50° C. was 2 hours. Primary drying and secondary drying was performed at −30° C. and 20° C. for 12 hours each. The temperature was ramped at 0.5° C./min. Chamber pressure during primary and secondary was 50 mT. After lyophilization was completed, lyophilizer chamber was backfilled to a half-atmospheric pressure with N2 and capped.

### Example 1

### pH Optimization Study

A multi-component buffer (MCB) consisting of 10 mM Sodium Phosphate, 10 mM Sodium Citrate, 10 mM Sodium Acetate, 10 mM L-Histidine and 125 mM Sodium Chloride was used to determine the optimal pH. This buffer system was intended to minimize counter ion (salt effects) that may have other wise had a greater effect than pH alone. The pH screening design matrix is shown in Table 2. IMC-A12 concentration was kept at 5 mg/mL. The pH range examined was 5.0-8.0, at 0.5 pH unit intervals. The effect of pH on thermal and mechanical stability was studied and the results presented below.

TABLE 2
Design matrix for pH screening

| Buffer | [A12], (mg/mL) | pH |
|---|---|---|
| MCB-1 | 5.0 | 5.0 |
| MCB-2 | 5.0 | 5.5 |
| MCB-3 | 5.0 | 6.0 |
| MCB-4 | 5.0 | 6.5 |
| MCB-4 | 5.0 | 7.0 |
| MCB-5 | 5.0 | 7.5 |
| MCB-6 | 5.0 | 8.0 |

Differential Scanning Calorimetry (DSC) Study

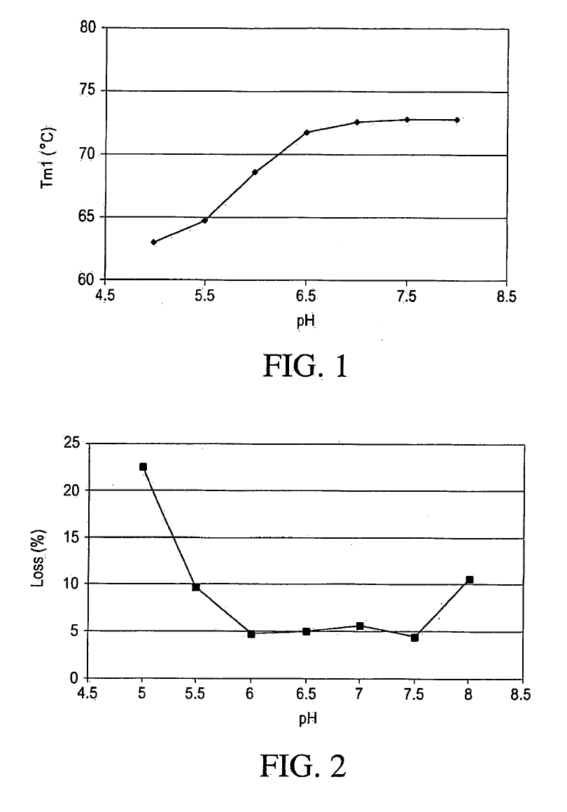

Thermal melting curves for IMC-A12 in experimental formulations (shown in Table 2) were assayed by Differential Scanning Calorimetry (DSC) in order to assess the transition temperature (Tm) for IMC-A12 in the test conditions. The protein concentration was 5 mg/mL and temperature ramping was from 5° C. to 95° C. at a scan rate of 1.5° C./min. The melting curves were fitted to a sum of three Tm. The melting temperature; Tm1, corresponding to first transition peak as a function of pH is shown in FIG. 1. Tm1 was comparable between pH 6.5-8.0.

Agitation Study

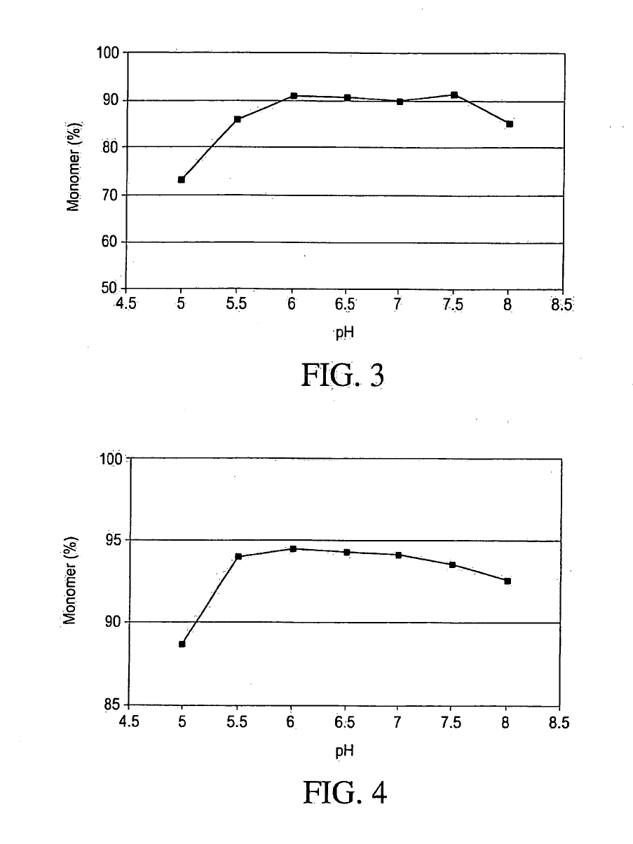

Samples were stressed by agitation on a platform shaker. Samples as described in Table 2 including 5 mL of IMC-A12 at 5 mg/mL in 27.5 mL glass vials were agitated at 300 RPM with Headspace set to be 81.8%. The study was performed at room temperature for 72 hours. The percent loss due to the formation of insoluble aggregate and percent monomer remaining as a function of pH is shown in FIGS. 2 and 3, respectively. Percent loss was least and percent monomer was highest between pH 6.0-7.0.

Real-Time Accelerated Temperature Stability at 40° C. and 50° C.

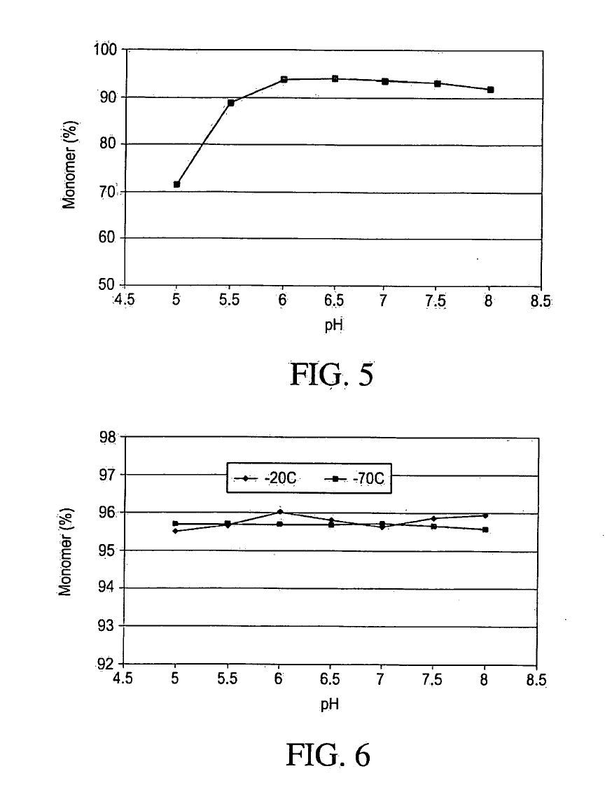

IMC-A12 at 5 mg/mL in various pH buffers (Table 2) was incubated at 40° C. for 3 weeks and at 50° C. for 1 week. The effect of pH on percent monomer was analyzed by SEC-HPLC. The variation of percent monomer remained as a function of pH after 3 week of incubation at 40° C. (FIG. 4) and 1 week of incubation at 50° C. (FIG. 5) are shown. The percent monomer remaining was largest between pH 6.0-6.5

Real-Time Freezing Temperature Stability at −20° C. and −70° C.

IMC-A12 at 5 mg/mL in various pH buffers (listed in Table 2) were incubated at −20° C. and −70° C. for three weeks. The effect of pH on percent monomer was analyzed by SEC-HPLC. The variation of percent monomer as a function of pH following three weeks of incubation is shown in FIG. 6. The pH did not have significant effect on percent monomer either at −20° C. or at −70° C.

Summary of pH Optimization

The optimal pH for IMC-A12 at 5 mg/mL was found to be between 6.0 and 6.5.

### Example 2

### Excipient Screening Study for Solution Formulations

The pH optimization studies in Example 1 demonstrated that IMC-A12 has greatest stability between pH 6.0 and 6.5. In this Example, we studied the effect of buffer type, citrate and histidine, on the stability of IMC-A12 at pH 6.0 and 6.5. Requirement for TWEEN 80 and NaCl and glycine concentration were also examined. Protein concentration was kept fixed at 5 mg/mL. The design matrix for excipient screening is shown in Table 3.

TABLE 3
Design matrix for excipient optimization

| | [A12], | Buffer | | Tween | | |
|---|---|---|---|---|---|---|
| | (mg/ | type | | 80 | [NaCl] | [Glycine] |
| Formulations | mL) | (10 mM) | pH | (%) | (mM) | (mM) |
| Formulation-1 | 5.0 | Histidine | 6.0 | 0 | 80 | 140 |
| Formulation-2 | 5.0 | Histidine | 6.0 | 0.01 | 100 | 75 |
| Formulation-3 | 5.0 | Histidine | 6.5 | 0.01 | 75 | 150 |
| Formulation-4 | 5.0 | Histidine | 6.5 | 0 | 150 | 0 |
| Formulation-5 | 5.0 | Histidine | 6.5 | 0.01 | 100 | 100 |
| Formulation-6 | 5.0 | Citrate | 6.5 | 0 | 150 | 0 |
| Formulation-7 | 5.0 | Citrate | 6.0 | 0.01 | 150 | 0 |
| Formulation-8 | 5.0 | Citrate | 6.0 | 0 | 50 | 150 |
| Formulation-9 | 5.0 | Citrate | 6.5 | 0.01 | 50 | 150 |
| Formulation-10 | 5.0 | Citrate | 6.5 | 0.01 | 100 | 100 |
| PBS | 5.0 | Phosphate | 7.2 | 0 | 145 | 0 |

Osmolality Measurement

The osmolality of the Table 3 formulations were measured using Wescor Vapor Pressure Osmometer. Results are shown in Table 4. Osmolality of tested formulations were within the desired range of 260-320 mOsmole/Kg.

TABLE 4
Osmolality of the formulations in Table 3

| | [A12], | Buffer type | | Tween 80 | [NaCl] | [Glycine] | Osmolality |
|---|---|---|---|---|---|---|---|
| Formulations | (mg/mL) | (10 mM) | pH | (%) | (mM) | (mM) | (mOsmole/Kg) |
| Formulation-1 | 5.0 | Histidine | 6.0 | 0 | 80 | 140 | 302 |
| Formulation-2 | 5.0 | Histidine | 6.0 | 0.01 | 100 | 75 | 283 |
| Formulation-3 | 5.0 | Histidine | 6.5 | 0.01 | 75 | 150 | 304 |
| Formulation-4 | 5.0 | Histidine | 6.5 | 0 | 150 | 0 | 304 |
| Formulation-5 | 5.0 | Histidine | 6.5 | 0.01 | 100 | 100 | 296 |
| Formulation-6 | 5.0 | Citrate | 6.5 | 0 | 150 | 0 | 318 |
| Formulation-7 | 5.0 | Citrate | 6.0 | 0.01 | 150 | 0 | 318 |
| Formulation-8 | 5.0 | Citrate | 6.0 | 0 | 50 | 150 | 275 |
| Formulation-9 | 5.0 | Citrate | 6.5 | 0.01 | 50 | 150 | 276 |
| Formulation-10 | 5.0 | Citrate | 6.5 | 0.01 | 100 | 100 | 316 |
| PBS | 5.0 | Phosphate | 7.2 | 0 | 145 | 0 | 294 |

Differential Scanning Calorimetry Study

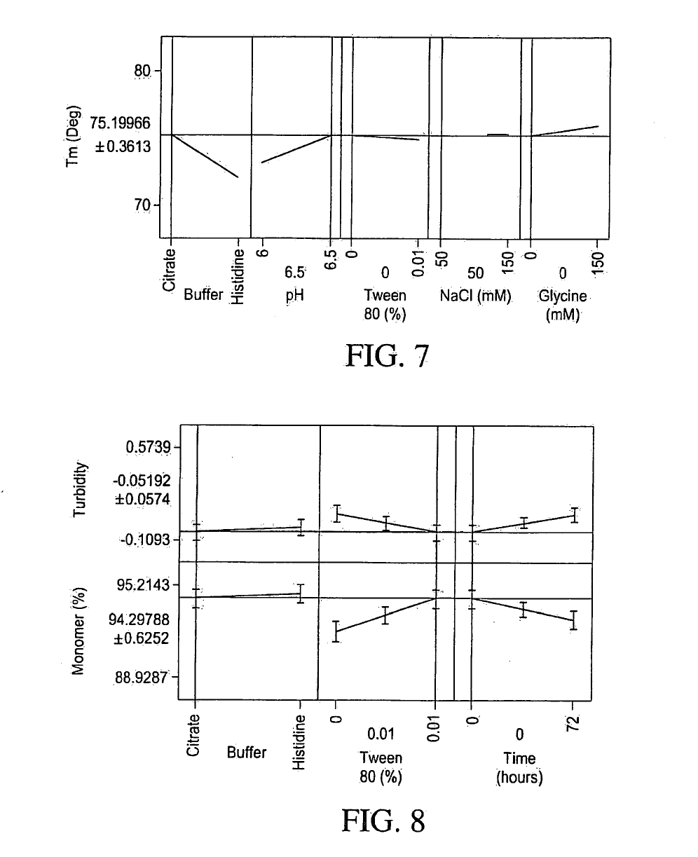

Thermal melting curves for IMC-A12 in experimental formulations (listed in Table 3) were assayed using DSC to assess the transition temperature (Tm) for IMC-A12 in the test conditions. The protein concentration was 5 mg/mL and temperature ramping was from 5° C. to 95° C. at a scan rate of 1.5° C./min. The melting temperature corresponding to the main transition peak was fitted to a linear regression model to estimate the effect of tested variables. The p and R_{sq} for the fit was 0.003 and 0.99, respectively. The prediction profiler for the effect of buffer type, pH, TWEEN 80 concentration, NaCl concentration, glycine concentration, on transition temperature is shown in FIG. 7. The optimal buffer was determined to be citrate buffer at pH 6.5. Glycine increased the melting temperature, and TWEEN 80 slightly lowered the melting temperature. NaCl does not have significant effect on the melting temperature.

Agitation Study

Samples were stressed by agitation on a platform shaker. The samples described in Table 3 with 5 mL of IMC-A12 at 5 mg/mL in 27.5 mL glass vial were agitated at 300 RPM. The study was performed at room temperature for up to 72 hours. Solution turbidity and percent monomer were determined as a function of agitation time. The effects of tested variables on turbidity and percent monomer were estimated by fitting the response to a linear regression model using JMP software. The p value for the Actual vs. Predicted plot for both turbidity and percent monomer were <0.001. The statistically significant variables were buffer, TWEEN and time. The prediction profiler for the effect of buffer, TWEEN 80, and time on turbidity and percent monomer is shown in FIG. 8. Citrate buffer with 0.01% TWEEN had the least turbidity and highest monomer content.

Real-Time Accelerated Temperature Stability at 40° C. and 50° C.

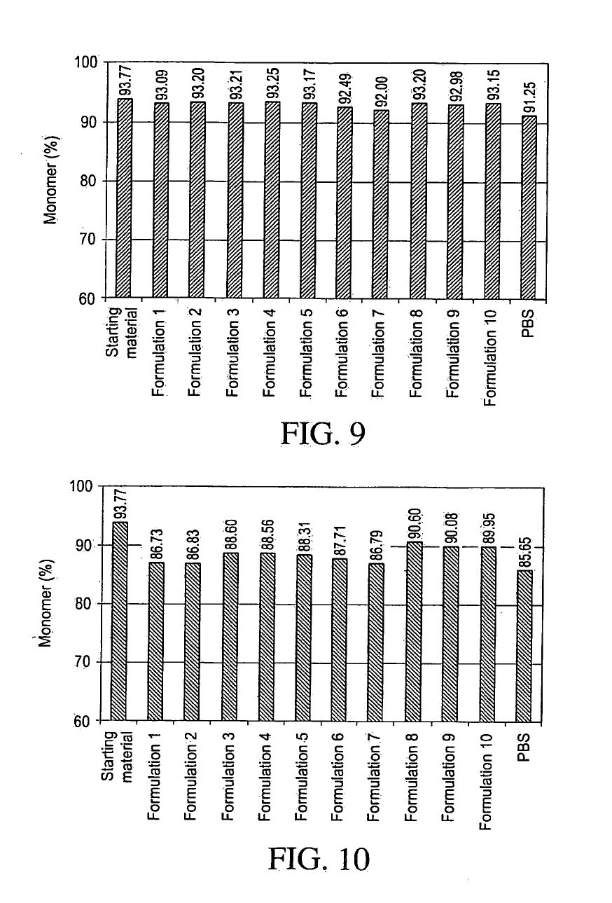

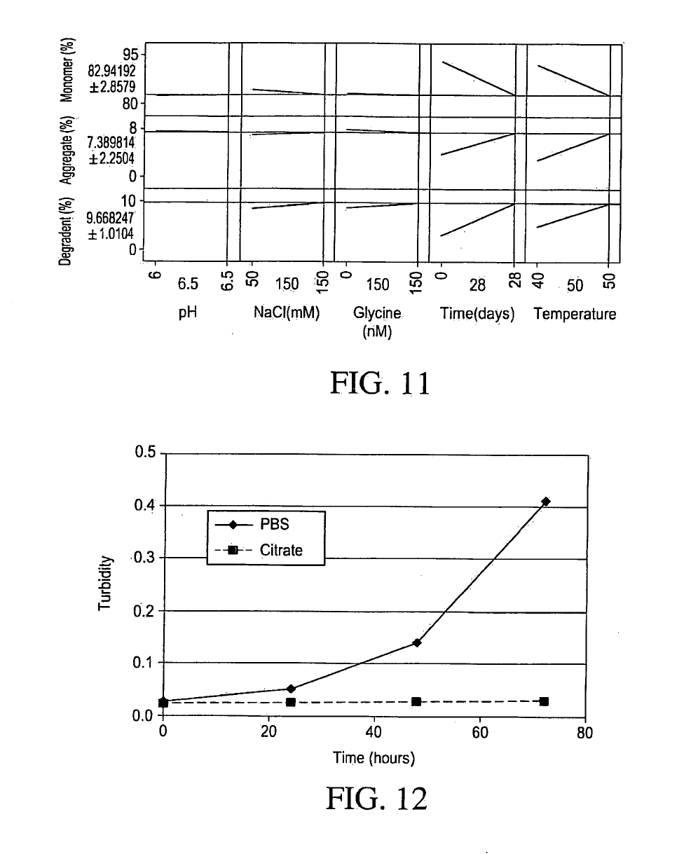

IMC-A12 at 5 mg/mL in the Table 3 formulations was incubated at 40° C. for 4 weeks and 50° C. for 2 weeks. Percent monomer for starting material and tested formulations after 4 weeks of incubation at 40° C., and 2 weeks of incubation at 50° C. are shown in FIGS. 9 and 10 respectively. DOE analyses of temperature stressed samples are also shown in FIG. 11. At 40° C., percent monomer for most of the tested formulations was comparable but better than PBS. At 50° C., formulations in citrate buffer (formulation 6-10) were superior to histidine buffers (formulation 1-5). DOE analysis in FIG. 11 shows that IMC-A12 has comparable stability between pH 6.0-6.5, and that NaCl has a destabilizing effect, while glycine has relatively less effect. Formulations 9 and 10 were found to be comparable. However, formulation 10 was preferred since it has less glycine concentration (closer to physiological condition).

Summary of Excipient Screening Study

DSC studies showed that Citrate buffer, glycine and pH 6.5 have increased IMC-A12 thermal stability. TWEEN-80 has slightly lowered the stability while NaCl did not have much effect. IMC-A12 is sensitive to mechanical stress. Thus, TWEEN 80 is required to stabilize against mechanical stress. IMC-A12 has better stability in citrate formulation than in Histidine at accelerated temperatures. Both, histidine and citrate buffers are superior than PBS formulation. Formulation 10, which contains 5 mg/mL, IMC-A12, 10 mM Citrate, 100 mM Glycine, 100 mM NaCl, 0.01% TWEEN 80, pH 6.5 (Citrate) was selected as an optimal formulation.

### Example 3

### Comparison Between PBS and Citrate Solution Formulations

As discussed above, we developed a new solution formulation for IMC-A12 that contains 5 mg/mL IMC-A12, 10 mM Sodium citrate, 100 mM Glycine, 100 mM NaCl, 0.01% TWEEN 80, at pH 6.5 (Citrate). In this Example, we compared the stability of IMC-A12 in Citrate formulation with a PBS formulation.

Agitation Study

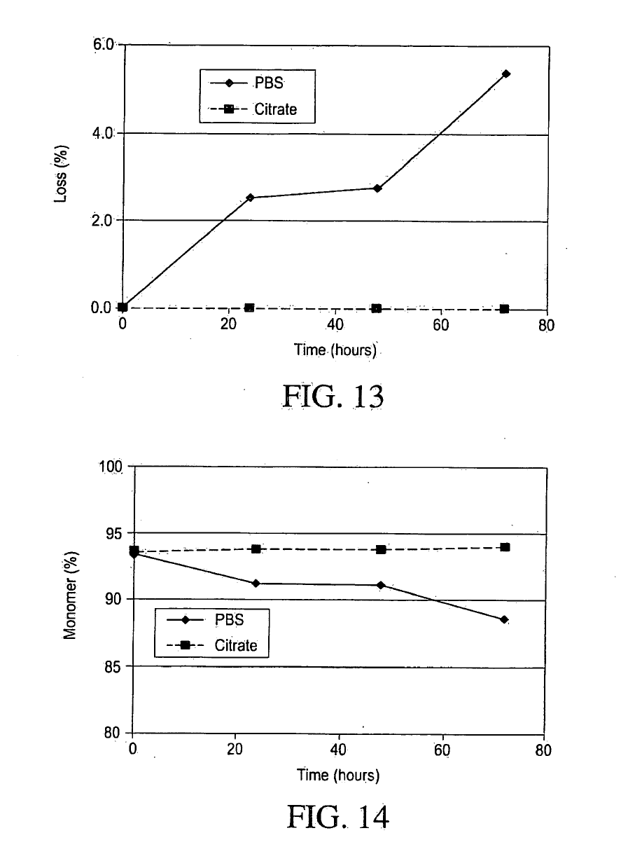

Samples were stressed by agitation on a platform shaker. The samples containing IMC-A12 at 5 mg/mL, in 27.5 mL glass vials were agitated at 300 RPM. The study was performed at room temperature for up to 72 hours. Concentration and turbidity measurements were performed using a Shimatzu 1601 biospec spectrophotometer. The concentration of IMC-A12 solutions was calculated from the absorbance at 280 nm, using an extinction coefficient of 1.5. Solution turbidity was measured by absorbance at 350 nm. The solution turbidity, percent material loss (due to the formation of insoluble aggregate), and percent monomer remaining as a function of agitation time are shown in FIGS. 12, 13 and 14, respectively. For PBS formulations of IMC-A12, solution turbidity and percent loss increased with agitation time, while percent monomer decreased. Turbidity, percent loss and percent monomer all remained unchanged for Citrate formulation.

Real-Time Accelerated Temperature Stability at 40° C.

IMC-A12 at 5 mg/mL in PBS or Citrate formulations were incubated at 40° C. for up to 3 months. Following incubation, samples were analyzed by SEC-HPLC, SDS-PAGE and IEF. Results are shown below.

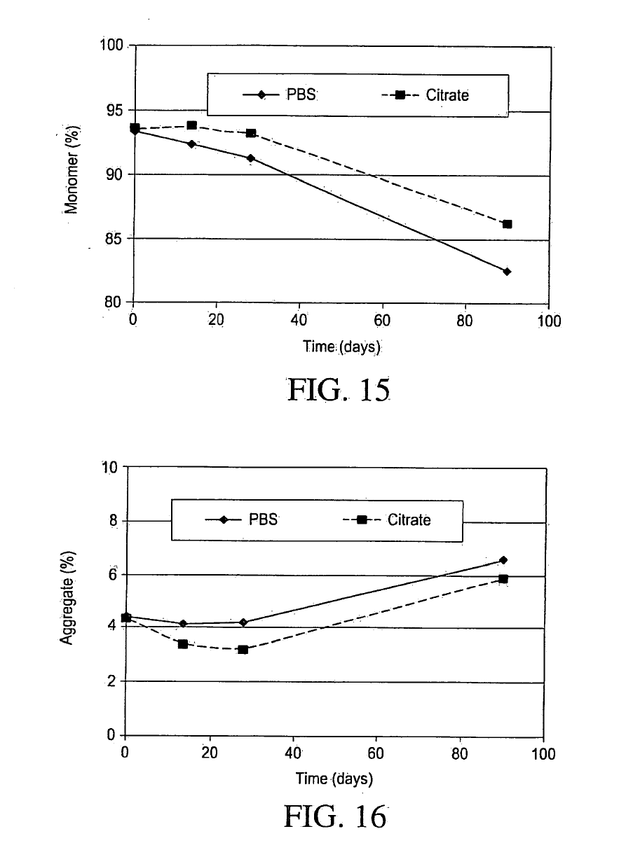

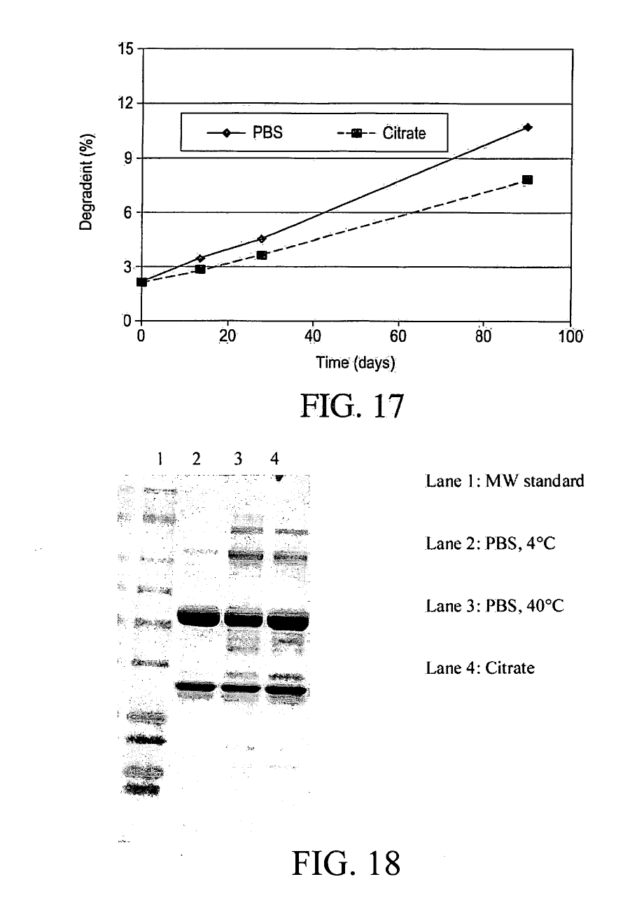

SEC-HPLC Analysis: Size exclusion chromatography was performed using an Agilent 1100 Series LC chromatograph and a Tosoh Biosep G3000SWXL column. The mobile phase was 10 mM Sodium phosphate, 0.5M CsCl pH 7.0. Fifty μg of sample was injected in a volume of 10 μl. The variation of percent monomer, aggregate, and degradent as a function of incubation time are shown in FIGS. 15, 16, and 17 respectively. Percent monomer decreased and percent aggregate and degradent increased for both the formulations, but the rate was slower for Citrate formulation compared to PBS formulation.

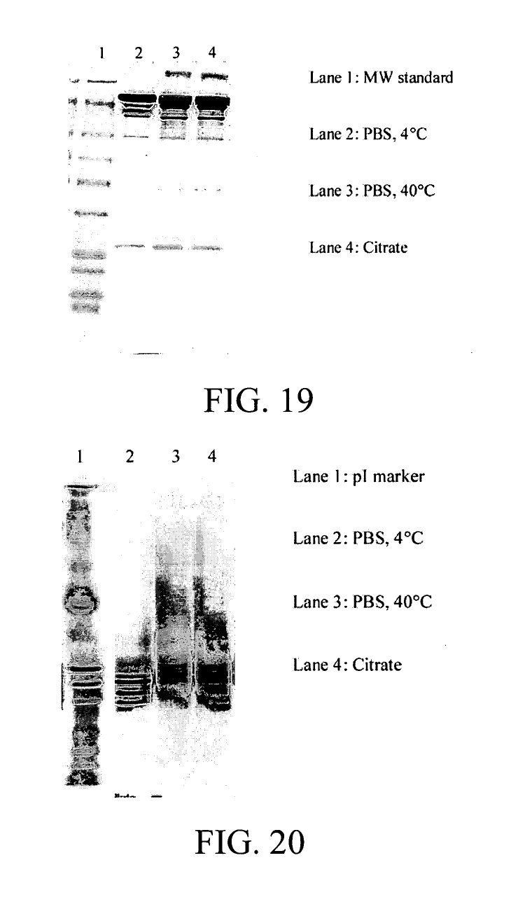

SDS-PAGE Analysis: IMC-A12 in PBS and Citrate formulations following 3 months of incubation at 40° C. was analyzed by reduced and non-reduced SDS-PAGE on 4-20% tris-glycine gradient gel. Ten μg of sample was loaded in a volume of 10 μl. Gel was stained with Coomassie blue. Results are shown in FIGS. 18 and 19, respectively. In comparison, more intense impurity bands were detected in the PBS formulation than in Citrate formulation.

IEF Analysis: Isoelectric, focusing (IEF) was performed using IsoGel® Agarose IEF plates with a pH range from 6.0 to 10.5. Test samples were buffer exchanged into miliQ water containing 0.5% TWEEN 80. The 10 μg sample was loaded in a volume of 10 μl. Gel was stained with Coomassie blue. IMC-A12 in PBS and Citrate formulations following 3 months of incubation at 40° C. was analyzed by IEF. Results are shown in FIG. 20. In comparison, more diffused and less defined bands were detected for PBS formulation than in Citrate formulation.

Freezing Temperature Stability of IMC-A12 at −20° C. and −70° C.

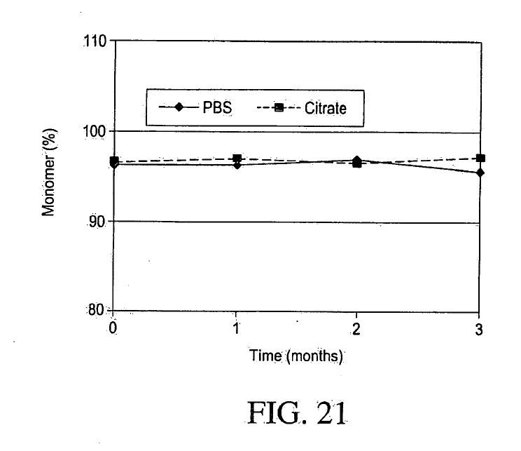

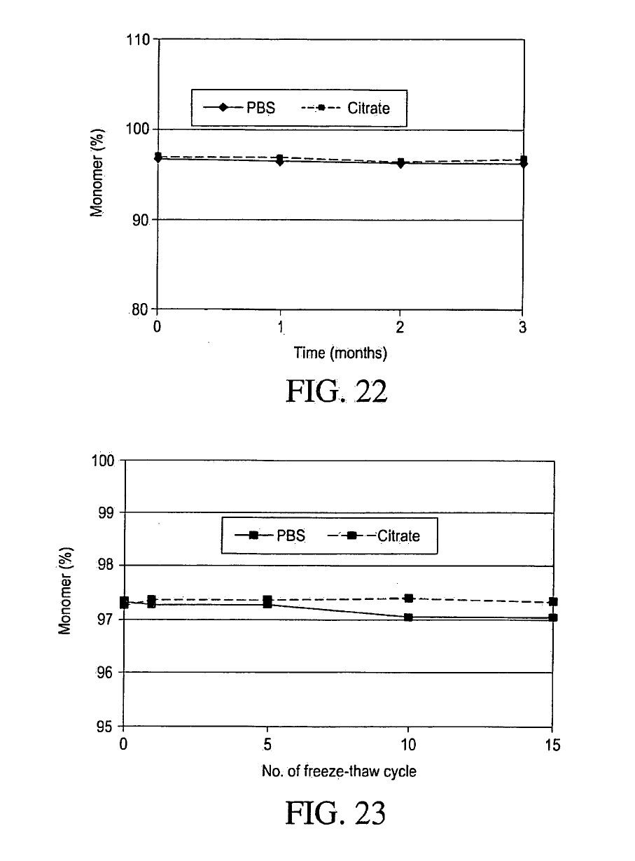

IMC-A12 at 5 mg/mL in PBS and Citrate formulations was incubated at −20° C. and −70° C. for up to 3 months. Percent monomer, following incubation was analyzed by SEC-HPLC. The variation of percent monomer as a function of time at −20° C. and at −70° C. are shown in FIGS. 21 and 22, respectively. The percent monomer did not change with time in either formulation.

Freeze-Thaw Stability of IMC-A12 at −20° C. and −70° C.

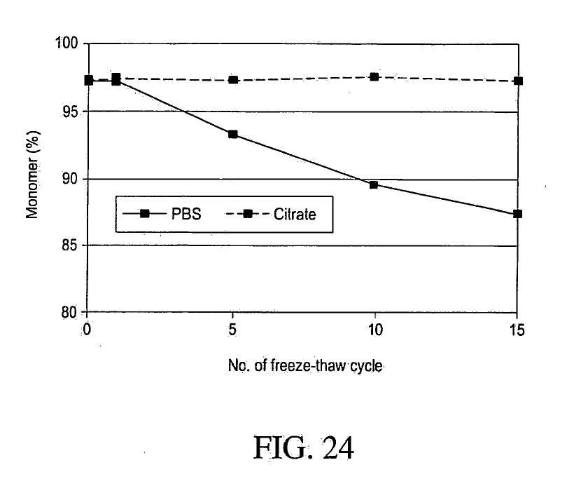

Freeze-thaw stability of IMC-A12 was evaluated by freezing the test sample to either −20° C. or −70° C. in a freeze-dryer (Lyo-star II, manufactured by FTS) with a ramp rate of 1° C./min. The sample was allowed to incubate for 1 hour and thawed at 4° C. with a ramp rate of 1° C./min. The freeze-thaw process was repeated up to 15 times. The variation of percent monomer as a function of number of freeze-thaw cycle at −20° C. and −70° C. are shown in FIGS. 23 and 24, respectively. As shown, IMC-A12 in Citrate formulation has better freeze-thaw stability than in PBS formulation. The decrease in percent monomer for PBS formulation was mainly due to increase in percent aggregates.

Photo-Stability of IMC-A12 Solution Formulations

Photo stability study for IMC-A12 was performed per ICH guideline. IMC-A12 at 5 mg/mL in PBS and Citrate formulations was exposed to light at room temperature. The total light exposure was 200 Watt hours/m2 near UV +1.2 million lux hours fluorescent. Control samples were wrapped with black paper to block light. Control and test samples were placed inside the photo stability chamber (Caron 6500 series, Caron, Marietta, Ohio). Following light exposure, both controls and test samples were analyzed by SEC-HPLC. Percent monomer, aggregate, and degradent for controls and light exposed samples are given in Table 5. IMC-A12 was found to be light sensitive in both formulations. However, the photo stability was significantly improved in the Citrate formulation than the PBS formulation.

TABLE 5
Photo-stability for IMC-A12 in PBS and Citrate Formulations.

| | | Monomer | Aggregates | Degradents |
|---|---|---|---|---|
| Formulations | Product lot | (%) | (%) | (%) |
| PBS-Control | 1278-116 | 96.6 | 1.5 | 2.0 |
| PBS-sample | 1278-116 | 73.5 | 22.8 | 3.7 |
| Citrate-Control | 1278-151 | 95.7 | 1.4 | 2.9 |
| Citrate-sample | 1278-151 | 81.9 | 14.2 | 3.9 |

Summary of Comparison between PBS and Citrate Formulations

IMC-A12 demonstrates significantly better stability in 10 mM Sodium citrate, 100 mM Glycine, 100 mM NaCl, 0.01% TWEEN 80, pH 6.5 (Citrate) formulation than in PBS formulation. Citrate is an isotonic formulation that is particulate free, stable against mechanical induced aggregation or precipitation, has minimized temperature-induced aggregation and degradation, is stabilized against freeze-thaw instability, and has enhanced photo stability.

### Example 4

### Screening of Buffers, Cryo- and -Lyo Protectents and Bulking Agents for Lyophilized Formulations

The buffer type, stabilizers and bulking agents for freeze-dried formulation was examined at IMC-A12 concentration of 20 mg/mL. The design matrix is shown in Table 6, fractional factorial design model was used. The design matrix for concentration optimization for IMC-A12, ratio of trehalose concentration to IMC-A12 concentration, and TWEEN 80 concentration is shown in Table 7. A mixture design model was used.

TABLE 6
Design matrix for stabilizers and bulking agents screening

| | | [A12] | | | | | |
|---|---|---|---|---|---|---|---|
| | Buffer | (mg/ | Trehalose | Sucrose | Mannitol | Glycine | |
| # | (10 mM) | mL) | (%) | (%) | (%) | (%) | pH |
| 1 | Histidine | 20 | 4 | 0 | 2 | 0 | 6.5 |
| 2 | Histidine | 20 | 0 | 2 | 3 | 0 | 6.5 |
| 3 | Histidine | 20 | 1 | 1 | 0 | 0 | 6.5 |
| 4 | Histidine | 20 | 0.5 | 3.5 | 0 | 2 | 6.5 |
| 5 | Histidine | 20 | 4 | 0 | 0 | 0 | 6.5 |
| 6 | Histidine | 20 | 0 | 4 | 0 | 0 | 6.5 |
| 7 | Citrate | 20 | 1.5 | 0.5 | 0 | 4 | 6.5 |
| 8 | Citrate | 20 | 0.5 | 1.5 | 4 | 0 | 6.5 |
| 9 | Citrate | 20 | 4 | 0 | 0 | 0 | 6.5 |
| 10 | Citrate | 20 | 0 | 4 | 0 | 0 | 6.5 |

TABLE 7
Design matrix for optimization of A12 concentration, Trehalose to A12
molar ratio, and TWEEN 80 concentration.

| | | | | Trehalose | Tween |
|---|---|---|---|---|---|
| | Buffer | | [A12], | to A12 | 80 |
| # | (10 mM) | pH | (mg/mL) | molar ratio | (%) |
| 1 | Histidine | 6.5 | 50.0 | 200 | 0.000 |
| 2 | Histidine | 6.5 | 10.0 | 1000 | 0.000 |
| 3 | Histidine | 6.5 | 10.0 | 200 | 0.010 |
| 4 | Histidine | 6.5 | 30.0 | 600 | 0.000 |
| 5 | Histidine | 6.5 | 10.0 | 600 | 0.005 |
| 6 | Histidine | 6.5 | 30.0 | 200 | 0.005 |
| 7 | Histidine | 6.5 | 22.9 | 460 | 0.003 |
| 8 | Histidine | 6.5 | 16.0 | 760 | 0.002 |
| 9 | Histidine | 6.5 | 10.0 | 440 | 0.007 |

For lyophilization, IMC-A12 was buffer exchanged into either neat 10 mM Histidine at pH 6.5, or 10 mM Citrate at pH 6.5 using Lab scale TFF and Pellicon® XL filter, 50K cut-off filter (Millipore, Corporation). Lyo and Cryo protectants were added from concentrated stock, after buffer exchange was done. Protein, concentration was determined by absorbance at 280 nm using an extinction coefficient of 1.50. TWEEN 80 was added from a 10% (w/v, in DI water) stock solution after protein concentration adjustments. All samples were filtered through 0.22 μm cutoff (Durapose PVDF membrane) syringe filter.

The buffer type, cryo- and lyo protectants and bulking agents were screened for effect on monomer, aggregate, degradent and turbidity of 20 mg/mL IMC-A12 in the formulations shown in Table 6. The lyophilized drug product was incubated at 40° C. and 50° C. for 3 months. Following incubation, lyophilized drug products were reconstituted into miliQ water to 5 mg/mL. Reconstituted products were analyzed by SEC-HPLC and Turbidity. The results were fitted using statistical software JMP. Results are summarized below.

Effects of Variables on Predicted Monomer, Aggregate, Degradent and Turbidity

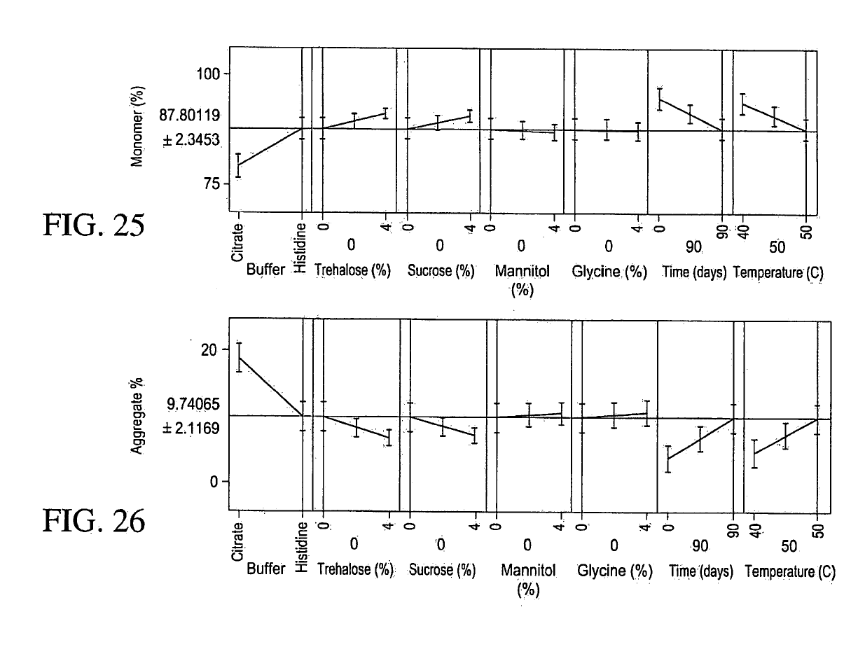

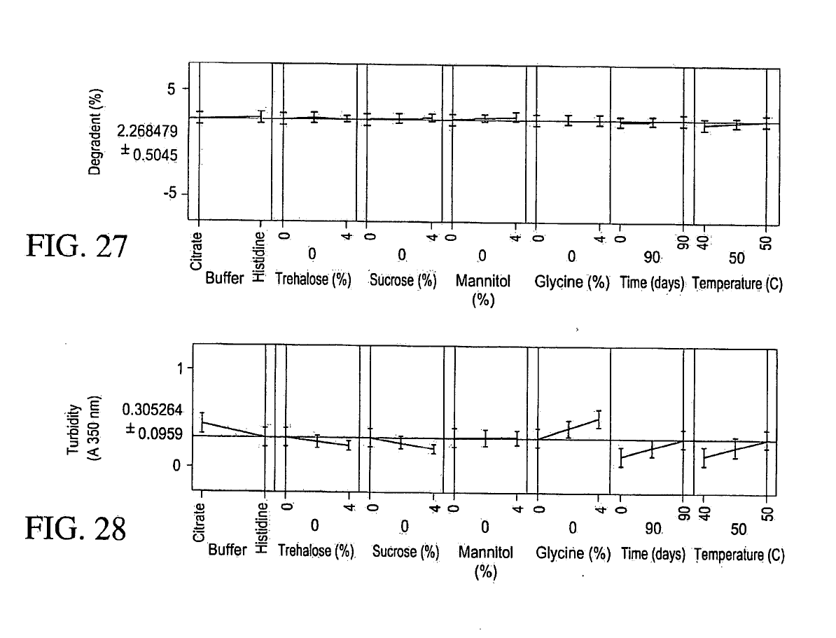

The reconstituted drug products were analyzed by SEC-HPLC and turbidity analysis. The variation of percent monomer, aggregate, degradent and turbidity as a function of buffer type, cryo-and-lyo protectants, and bulking agents are shown in FIGS. 25, 26, 27, and 28, respectively. The results demonstrated that (1) Histidine buffer causes greater monomer and lesser aggregate than Citrate buffer. (2) Trehalose and Sucrose increase the monomer content and lower the aggregation. (3) The bulking agents, mannitol and glycine did not have significant effects of the percent monomer or aggregate. None of the tested variables has significant effect on degradent.

Confirmation of Predicted Results by One-Factor-at-a-Time Approach

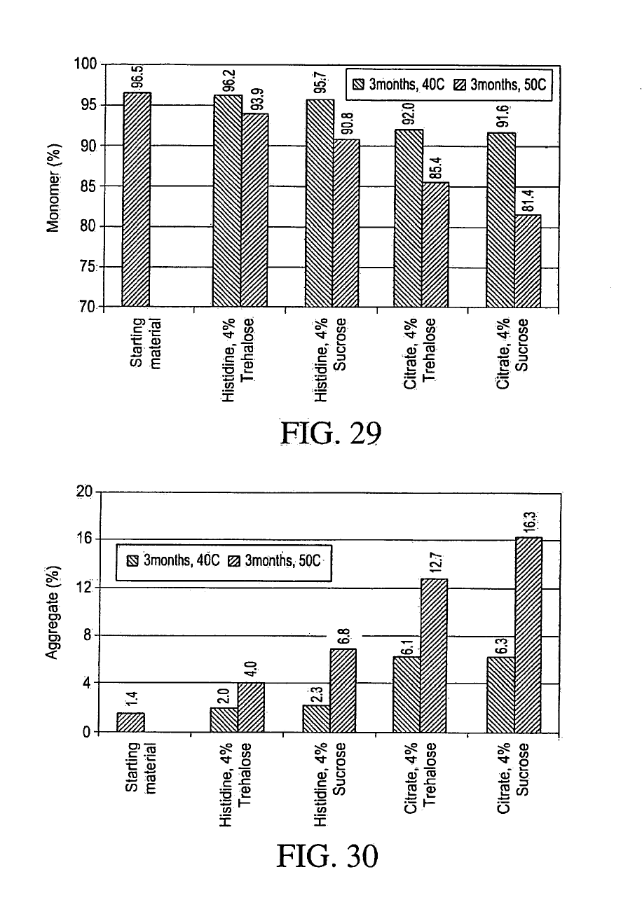

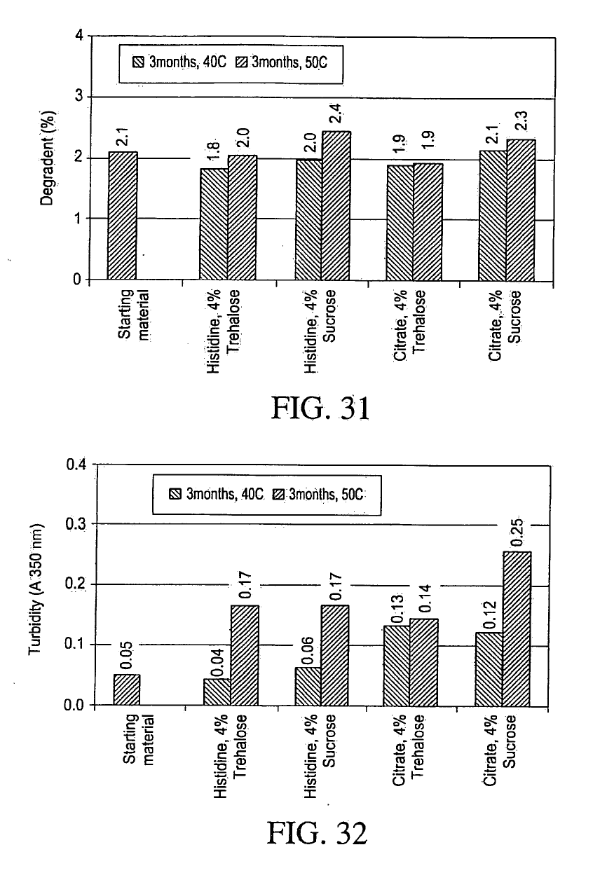

In order to confirm statistical predicted results, the formulations 5, 6, 9 and 10 in Table 6 were analyzed using one-factor-at-a-time approach. The effect of incubation at 40° C. and 50° C. for up to 3 months on the percent monomer, aggregate, degradent and turbidity, are shown in FIGS. 29, 30, 31, and 32, respectively. Results confirmed that (1) histidine is a superior buffer than citrate and (2) thehalose is a better stabilizer than sucrose.

Summary of Buffer Type, Cryo- and Lyo-Protectant, and Bulking Agent Screening

Freeze-dried IMC-A12 formulations have greater stability in histidine buffer than citrate buffer. Trehalose has better stabilizing effect than sucrose. The presence of the bulking agents, mannitol and glycine, did not significantly effect stability.

### Example 5

### Optimization for IMC-A12, Trehalose and TWEEN 80 Concentration for Optimal Freeze-Dried Formulation

The mixture design model was used to optimize the IMC-A12 concentration, ratio of thehalose:IMC-A12, and concentration of TWEEN 80 for optimal formulation. The experiment design matrix is shown in Table 7. The lyophilized IMC-A12 was incubated at 4° C., 40° C. and 50° C. for up to 4 months. Results are discussed below.

Variation of Percent Monomer as a Function of Formulation

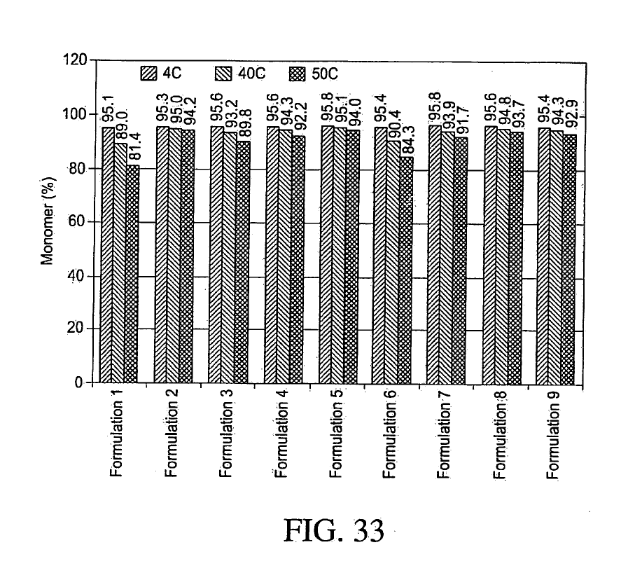

Lyophilized IMC-A12 formulations from Table 7 were incubated at 4° C., 40° C. and 50° C. for up to 4 months. The lyophilized samples were reconstituted with MiliQ water to 5 mg/mL. The reconstituted samples were analyzed by SEC-HPLC to determine the remaining monomer percent. The results are shown in FIG. 33.

Effect of IMC-A12 Concentration, Ratio of Trehalose:A12 and TWEEN 80 Concentration on Rate of Monomer Change

The rate of monomer change was defined as a slope of monomer variation as a function of time. The Excel software was used to calculate the slope. The rate of monomer change was smallest at lowest IMC-A12 concentration and at highest trehalose to IMC-A12 ratio. TWEEN 80 did not have significant effect.

Summary Of Optimization Study

Predicted monomer content increased with decrease of IMC-A12 concentration and increase of Trehalose to IMC-A12 ratio. At fixed IMC-A12 concentration, monomer content increased by increasing trehalose to IMC-A12 ratio TWEEN 80 had minimal effect on percent monomer. Formulation 4 that has 30 mg/mL IMC-A12 and trehalose to IMC-A12 ratio of 600 was selected as a preferred formulation.

### Example 6

### Characterization of Freeze-Dried IMC-A12

The moisture content of the lyophilized product as determined by Karl-Fisher analysis was found to be ˜1.0%. The freeze-dried IMC-A12 was reconstituted to 5 mg/mL with miliQ water. Reconstitution time was about 1-2 min.

Effect of Lyophilization on IMC-A12 Stability

To ensure that the lyophilization process had not changed the IMC-A12 stability, the IMC-A12 was analyzed by SEC-HPLC before and after lyophilization. Lyophilized IMC-A12 was reconstituted prior to SEC-HPLC analysis. The percent monomer, aggregate and degradent for pre and post lyophilized A 12 are shown in Table 8.

TABLE 8
SEC-HPLC Analysis of Pre and Post Lyophilized IMC-A12

| | Monomer | Aggregate | Degradent |
|---|---|---|---|
| | (%) | (%) | (%) |
| Before lyophilization | 95.7 | 3.0 | 1.4 |
| After lyophilization | 95.6 | 3.1 | 1.5 |

Effect of Lyophilization on Conformational Stability of IMC-A12

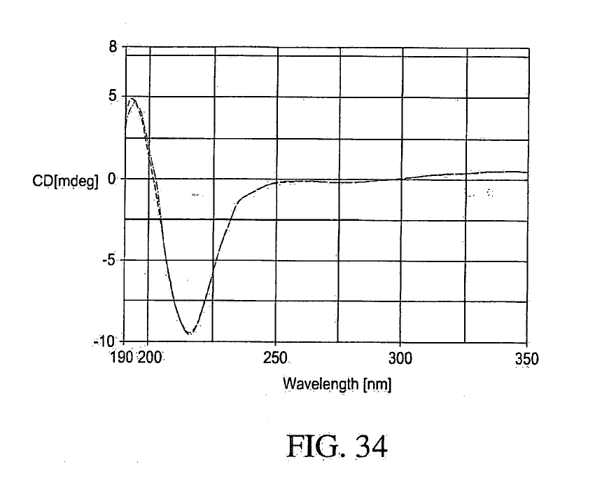

To ensure that the lyophilization process has not altered the secondary structure of A12, secondary structure of pre and post lyophilized IMC-A12 was examined by circular dichorism. The CD spectrums were collected using a Jasco 810 circular dichorism spectrophotometer, the IMC-A12 concentration was 0.1 mg/mL. The CD spectra before lyophilization and after lyophilization and reconstitution are shown in FIG. 34. Secondary structure of IMC-A12 was not altered because of lyophilization.

Effect of Lyophilization on Particulate Counts for IMC-A12

The effect of lyophilization on particulate content for IMC-A12 was measured using HIAC ROYCO MODEL 9703 Liquid Particle System. IMC-A12 before and after lyophilization was diluted/reconstituted to 5 mg/mL. Results are shown in Table 9. The particulate counts did not changed significantly.

TABLE 9
HIAC Analysis of Pre and Post Lyophilized IMC-A12

| | =10 μm/mL | =25 μm/mL | =50 μm/mL |
|---|---|---|---|
| Before lyophilization | 26.33 | 1.67 | 0.00 |
| After lyophilization | 38.67 | 0.33 | 0.00 |

### Example 7

### Comparison between Solution and Lyophilized IMC-A12 Formulations

The following formulations were compared:

(1) PBS Solution formulation, 5 mg/mL IMC-A12 in PBS

(2) Citrate Solution formulation, 5 mg/mL IMC-A12 in 10 mM Sodium citrate, 100 mM NaCl, 100 mM Glycine, 0.01% TWEEN 80 (w/v), pH 6.5

(3) Lyophilized formulation, 30 mg/mL IMC-A12, 10 mM L-Histidine, 4.6% Trehalose, pH 6.5

Real-time Accelerated Temperature Stability

The PBS and Citrate solution formulations, and the lyophilized formulation were incubated at 4° C., 40° C., 50° C. The lyophilized IMC-A12 was reconstituted to 5 mg/mL with milli-Q water prior to analysis. The solution and reconstituted lyophilized formulations were analyzed by SEC-HPLC and SDS-PAGE.

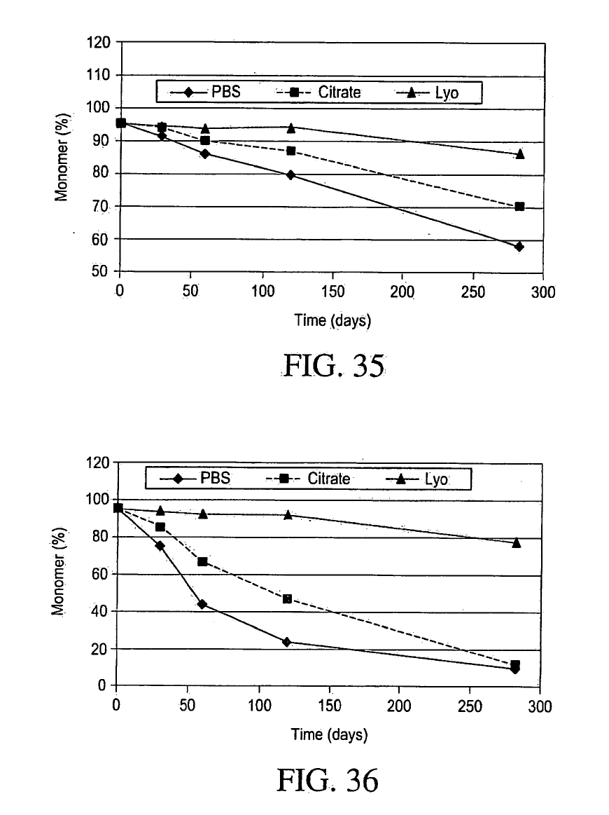

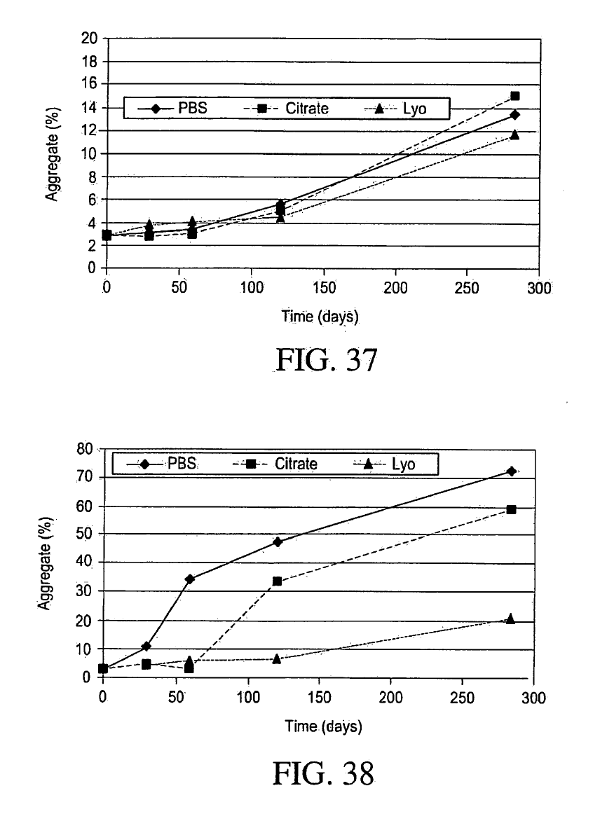

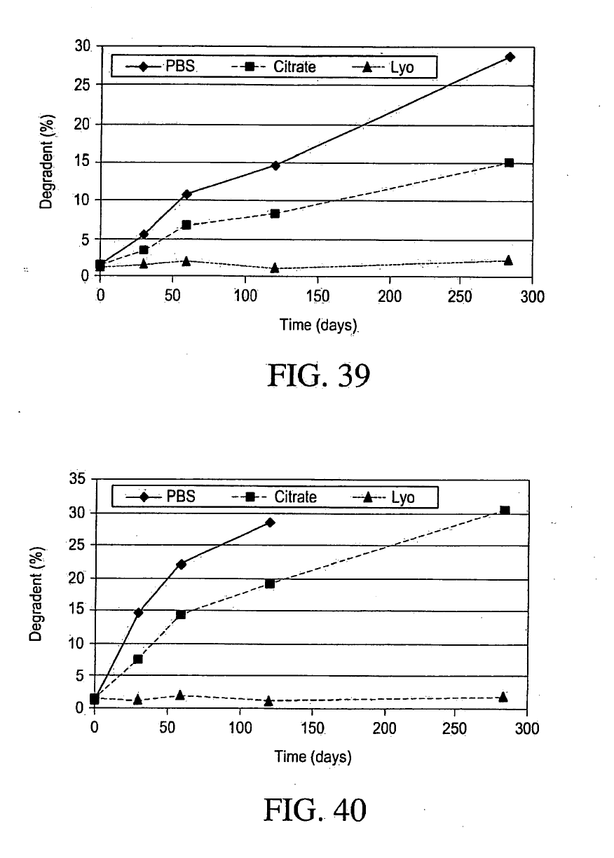

IMC-A12 solution formulations in PBS and citrate buffer, and IMC-A12 in the preferred lyophilized formulation were incubated at 40° C. and 50° C. for 4 months. The lyophilized samples were reconstituted in Milli-Q water and percent monomer was analyzed by SEC-HPLC. The percent monomer after 4 month incubation at 40° C. and 50° C. is shown in FIGS. 35 and 36, respectively. The percent aggregate after 4 month incubation at 40° C. and 50° C. is shown in FIGS. 37 and 38, respectively. The percent degradent after 4 month incubation at 40° C. and 50° C. is shown in FIGS. 39 and 40, respectively.

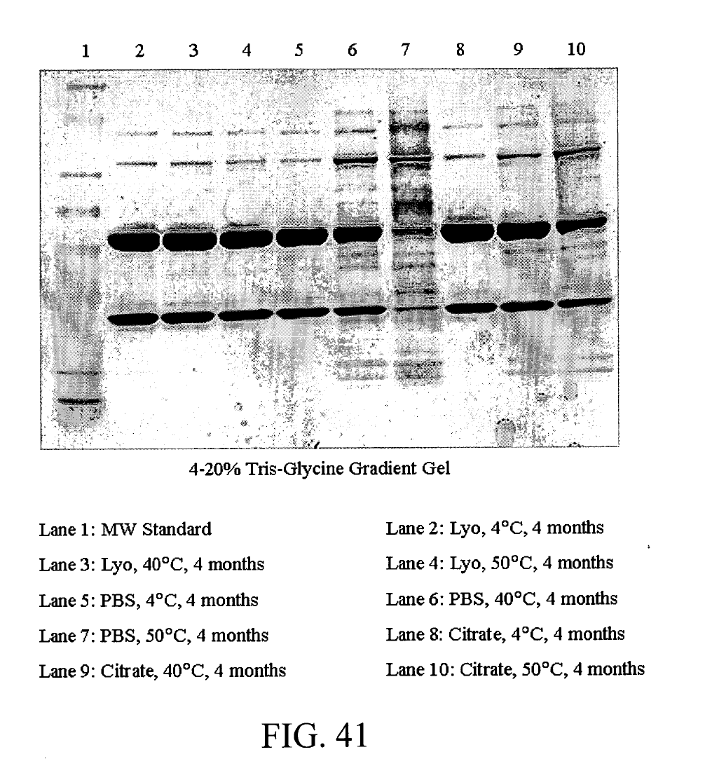

SDS-page (reduced) analysis of the samples after incubation at 4° C., 40° C. and 50° C. for 4 months is shown in FIG. 41. IMC-A12 solution formulations in PBS and citrate buffer, and IMC-A12 in the preferred lyophilized formulation were incubated at 4° C., 40° C. and 50° C. for 4 months. The lyophilized samples were reconstituted in Milli-Q water and 10 us were loaded into a 4-20% Tris-glycine gel. The gel was stained with Coomassie blue.

Photo-stability of Lyophilized Formulation

Photo stability was performed as described above. The lyophilized IMC-A12 and solution formulations PBS and Citrate were exposed to light at room temperature. The total light exposure was 200 Watt hours/m2 near UV +1.2 million lux hours fluorescent. Controlled samples were wrapped with black paper to block light. Control and test samples were placed inside the photo stability chamber (Caron 6500 series, Caron, Marietta, Ohio). Following light exposure, both controls and test samples were analyzed by SEC-HPLC. Percent monomer, aggregate and degradent for controls and light exposed samples are given in Table 10. IMC-A12 was found to be light sensitive in both the formulation; however, the photo stability was significantly better in the citrate formulation than the PBS formulation.

TABLE 10
Photo-stability Study for IMC-A12 in Lyophilized and
Solutions Formulations.

| | Monomer | Aggregates | Degradents |
|---|---|---|---|
| Formulation | (%) | (%) | (%) |
| PBS-Control | 96.6 | 1.5 | 2.0 |
| PBS-Light Exposed | 73.5 | 22.8 | 3.7 |
| Citrate-Control | 95.7 | 1.4 | 2.9 |
| Citrate-Light Exposed | 81.9 | 14.2 | 3.9 |
| Lyophilized-Control | 98.1 | 1.0 | 0.9 |
| Lyophilized-Light Exposed | 94.0 | 4.6 | 1.4 |

1-20. (canceled)

21. A liquid formulation comprising IMC-A12 or IMC-2F8, a pharmaceutically acceptable buffer at a pH ranging from about 6.0 to about 7.0, a pharmaceutically acceptable salt, a pharmaceutically acceptable stabilizing agent, and a pharmaceutically acceptable surfactant.

22. The liquid formulation of claim 21 wherein IMC-A12 or IMC-2F8 concentration ranges from about 5 mg/ml to about 30 mg/ml.

23. The liquid formulation of claim 21 wherein the buffer is an organic buffer selected from the group consisting of histidine, citrate, malate, tartrate, succinate, and acetate buffers, in a concentration ranging from about 5 mM to about 50 mM.

24. The liquid formulation of claim 23 wherein the buffer is at a concentration of about 10 mM.

25. The liquid formulation of claim 24 wherein the buffer is a citrate buffer.

26. The liquid formulation of claim 21 wherein the stabilizing agent is selected from the group consisting of aspartic acid, lactobionic acid, glycine, trehalose, mannitol, and sucrose.

27. The liquid formulation of claim 26 wherein the stabilizing agent ranges in concentration from about 75 mM to about 150 mM.

28. The liquid formulation of claim 27 wherein the stabilizing agent concentration is about 100 mM.

29. The liquid formulation of claim 28 wherein the stabilizing agent is trehalose or glycine.

30. The liquid formulation of claim 29 wherein the stabilizing agent is glycine.

31. The liquid formulation of claim 21 wherein the salt is NaCl in a concentration ranging from about 75 to about 150 mM.

32. The liquid formulation of claim 31 wherein the NaCl is at a concentration of about 100 mM.

33. The liquid formulation of claim 21 wherein the surfactant is selected from the group consisting of polysorbate 20, polysorbate 80, polyethylene-polypropylene glycol, and bile salts, in a concentration ranging from about 0.001% to about 1.0% (weight per volume).

34. The liquid formulation of claim 33 wherein the surfactant is at a concentration of about 0.01% (weight per volume).

35. The liquid formulation of claim 21 wherein the pH ranges from about 6.0 to about 6.5.

36. The liquid formulation of claim 35 wherein pH is about 6.5.

37. The liquid formulation of claim 21 that comprises about 5 mg/ml IMC-A12 antibody, about 10 mM sodium citrate buffer, about 100 mM glycine stabilizer, about 100 mM NaCl, and about 0.01% polysorbate 80, wherein said liquid formulation is at a pH of about 6.5.

38. The liquid formulation of claim 21 that has been lyophilized to form a lyophylized formulation.

39. A lyophilized formulation comprising 30 mg/ml IMC-A12, about 10 mM histidine buffer and about 4.6% trehalose, wherein upon reconstitution the formulation has a pH of 6.5.


SEQUENCE LISTING (6 sequences)

SEQ ID NO: 1 - DNA, 1383 bp, Homo sapiens
  CDS (1)..(1383)
gaggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgagagcgccattacgatttttggagtggtccacccaagaccactactactactactacatggacgtctggggcaaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 460 aa, Homo sapiens
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 645 bp, Homo sapiens
  CDS (1)..(645)
tcttctgagctgactcaggaccctgctgtgtctgtggccttgggacagacagtcaggatcacatgccaaggagacagcctcagaagctattatgcaacctggtaccagcagaagccaggacaggcccctattcttgtcatctatggtgaaaataagcggccctcagggatcccagaccgattctctggctccagctcaggaaacacagcttccttgaccatcactggggctcaggcagaagatgaggctgactactattgtaaatctcgggatggcagtggtcaacatctggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctgcagaatgctcttga

SEQ ID NO: 4 - protein, 214 aa, Homo sapiens
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 5 - DNA, 645 bp, Homo sapiens
  CDS (1)..(645)
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacaacagtgataaccgtctgatatttggcggcgggaccaagctgaccgtcctcagtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctgcagaatgctcttga

SEQ ID NO: 6 - protein, 214 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDNSDNRLIFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS